(19) United States
(12) Patent Application Publication
(10) Pub. No.: US 2014/0349885 A1
(43) Pub. Date: Nov. 27, 2014

(54) ENHANCEMENT OF SENSITIVITY AND SPECIFICITY OF KETOSTEROIDS AND KETO OR ALDEHYDE CONTAINING ANALYTES

(71) Applicant: DH Technologies development Pte. Ltd., Singapore (SG)

(72) Inventors: Subhakar Dey, Lexington, MA (US); Subhasish Purkayastha, Acton, MA (US)

(21) Appl. No.: 14/368,358

(22) PCT Filed: Dec. 6, 2012
(86) PCT No.: PCT/IB2012/002619

§ 371 Date: Jun. 24, 2014

Related U.S. Application Data
(60) Provisional application No. 61/583,441, filed on Jan. 5, 2012.

(51) Int. Cl. G01N 33/74; G01N 33/58

(52) U.S. Cl. CPC G01N 33/743; G01N 33/58; G01N 2458/15; G01N 2560/00
USPC 506/12; 436/71; 436/128; 506/15

(57) ABSTRACT

A method, a labeling reagent, sets of labeling reagents, and labeling techniques are provided for the relative quantitation, absolute quantitation, or both, of ketone or aldehyde compounds including, but not limited to, analytes comprising steroids or ketosteroids. The analytes can be medical or pharmaceutical compounds in biological matrices. Methods for labeling, analyzing, and quantifying ketone or aldehyde compounds are also disclosed as are methods that also use mass spectrometry.

### RELATED APPLICATIONS

This application claims the benefit and priority from U.S. Provisional Application Ser. No. 61/583,441, filed on Jan. 5, 2012, the entire contents of which is hereby incorporated by reference herein.

### FIELD

Present teachings relate to the enhancement of sensitivity and specificity of analytes containing keto or aldehyde functionalities including ketosteroids and by site specific derivatization and targeted selection of signature ion using a liquid chromatography-mass spectrometry-mass spectrometry workflow.

### BACKGROUND

The ketones and aldehydes are polar chemical functionalities having a carbonyl group linked to one or two other carbon atoms. Ketone and aldehydes compounds, play an important role in industry, agriculture, and medicine. Ketones and aldehydes are also important agents in human metabolism and biochemistry. Ketosteroids, in particular, are a class of ketone-containing steroid compounds and are uniquely valuable in research and clinical diagnosis because these compounds are critical agents in hormone-regulated biological processes and have strong biological activity at very low concentrations. Many ketosteroids are also potentially valuable pharmaceutical agents and the analysis of their function and metabolism in the body are useful in both medical treatments and diagnostic techniques for the detection of disease.

Analysis and measurement of ketone and aldehyde compounds is challenging because these compounds can be present at low levels in clinical and biological samples such as plasma. Standard chromatographic techniques such as GC-MS methods for analysis after chemical derivatization are available. See, e.g., Song, J. et al., Journal of Chromatography B., Vol. 791, Issues 1-2, (127-135) 2003. Methods using fluorescence detection are available and some specific immunoassays, including radioimmunoassays (RIAs), are available, but these usually do not offer multi-component analysis. The major problems with RIAs are lack of specificity and the need to perform a different assay for each steroid.

Examples in the literature of LC/MS strategies exploit derivatization of the analytes, but the ionization efficiency is relatively low and these strategies have failed to achieve the limit of detection required for the assay to be viable in a clinical setting using mass spectrometry and also lack multiplexing capability. Steroid analysis in biological samples is also crucial for the evaluation and clinical detection of various endocrine and metabolic disorders. Clinical laboratories are currently performing radioimmunoassays (RIAs) for high throughput screening of steroids.

The above challenges posed by attempting to measure ketone and aldehyde compounds in samples are also magnified by the desire to rapidly screen and/or analyze a large number of biological samples for the specific compounds of interest or a panel of ketone or aldehyde analytes. Although mass spectrometry can provide rapid throughput, the use of mass spectrometry for identifying and quantifying ketone and aldehyde steroids are particularly challenging because of poor ionization efficiency, complex ionization patterns, interference in the mass measurement by isobaric compounds and low sample concentrations in the sample medium. In addition, the highly hydrophobic ketosteroids pose chromatographic challenge when using Reversed Phase (RP) chromatography in LC/MS/MS.

Therefore, although techniques for rapid and efficient analysis and quantitation of ketone and aldehyde compounds are highly desirable because of the biological importance of these compounds, the existing techniques are not ideal due to lack of sensitivity, cross-reacting substances, and other challenges inherent in the chemistry of the compounds.

Sensitive, selective, and accurate analysis of ketosteroids can be used for the monitoring of abnormal adrenal functions. The ionization efficiency of native ketosteroids in positive MS/MS can be poor, resulting often times in insufficient limits of detection (LODs), especially when analyzing human samples from infants and children. Derivatization of ketosteroids via their keto functionality to form oximes has been used to improve ionization and enhance sensitivity, as described, for example, in Kushnir et al., Performance Characteristics of a Novel Tandem Mass Spectrometry Assay for Serum Testosterone, Clin. Chem. 52:1, 120-128, 2006, which is incorporated herein in its entirety by reference.

MRM analysis and MS/MS conditions work well in clean solvent, however, when using complex biological samples, a high background (BKG) noise, often from the same mass Q1/Q3 interfaces, can be produced, complicating chromatography and reducing detection limits. A need exits for a method for sensitive and specific quantitation of ketosteroids and other analytes containing a keto or aldehyde functionality.

### SUMMARY

In accordance with one broad aspect of the teachings, certain embodiments relate to a method of operating a mass spectrometer system. This method provides highly sensitive and specific analysis (higher signal to noise ratios) of ketones and aldehydes with very low background noise in MS/MS.

In some embodiments, there is provided herewith a method for mass analysis of an analyte from a biological matrix comprising: derivatizing an analyte comprising an aldehyde or ketone functional group, with a labeling reagent of formula (I):

Y—(CH₂)ₙ—O—NH₂ (I)

where n is 2, 3, 4, 5, or 6 and Y is:

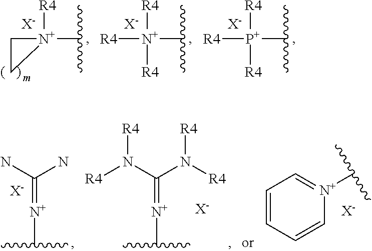

wherein each R₄ is independently H or a C₁-C₁₈ alkyl which is branched or straight chain,
m is an integer between 1 and 20, and
X is an anion,
   or a salt or hydrate thereof, to form a labeled analyte; ionizing the labeled analyte at a low to high collision energy so as to produce predominant signature ion fragments; and detecting the signature ion fragments by mass analysis. In some embodiments, the collision energy can be, for example, less than about 65 ev, such as in a range of 30 to 130 ev.

In some embodiments, the labeling reagent is:

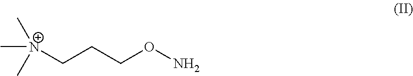

The predominant signature ion fragment can be, for example, a neutral loss fragment or a neutral loss comprising a structural fragment of the analyte and the labeling reagent, or a part thereof. In some embodiments, there are more than one predominant signature ion fragments. In some embodiments, there are 2, 3, 4, or 5 predominant signature ion fragments.

The method can also comprise the step of extracting the analyte using either liquid-liquid extraction, solid-liquid-extraction or protein precipitation using hydrophobic solvents prior to the derivatizing step. Alternatively a step of subjecting the analyte to chromatographic separation prior to the derivatizing step.

In some embodiments, the analyte is a ketosteroid. Analysis of such compounds from a biological matrix, such as blood, serum, plasma, urine, or saliva is within the scope of the present teachings.

In some embodiments, the signature ion fragment comprises:

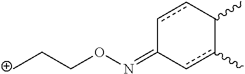

wherein each [symbol] is either a single or double bond and each [symbol] is either absent or indicates one or more bonds. When the analyte is a testosterone or testosterone derivative, the signature ion fragments can comprise, in some embodiments, a fragment ion having a mass of 164.2 and a second fragment ion having a mass of 152.2. Similarly, when the analyte is progesterone or progesterone derivative, the signature fragment ion can have a mass of 312.2.

Thus, several aspects provide for the reduction or elimination of background noise by using the derivatization chemistry of ketosteroids with permanently charged aminoxy reagent (Quaternary Aminoxy) and targeted fragmentation that can comprise both the reagent and the backbone of the derivatized compound. The derivatization with readily ionized/ionizable molecule can result in better ionization efficiency in, for example, ESI/MS/MS, which can increase the sensitivity and detection of the analytes of interest. When the fragment ion (Q3 signature ion) is carefully selected to comprise structural fragments with attached derivatization reagent (or part of the reagent), both the sensitivity and selectivity can be enhanced. The chances that a compound with the same Q1/Q3 transition would be detected and create BKG noise interference are very low.

In some embodiments, ketosteroid analysis kits can be provided to enable highly sensitive (low pg/mL concentrations) quantitation of ketosteroids from complex biological matrices.

The present teachings provide for the separation and characterization of compounds that cannot be readily separated and analyzed, such as isobaric ketosteroids in a biological sample, such as testosterone (Te) and epi-testosterone (epi-Te). These compounds can undergo the same fragmentation pattern in MS/MS, thus chromatographic separation can be necessary.

In some embodiments, the methods described herein can measure relative concentration, absolute concentration, or both, and can be applied to one or more ketones or aldehydes such as steroids containing a ketone or aldehyde group in one or more samples. The present methods can use an isotopically enriched Internal Standard (IS) or an isobaric labeling reagents, as well as mass differential labeling reagents, depending on the selection of isotopic substitution and labeling strategies for the compounds for the detection of ketosteroids.

In some embodiments, the present methods can quantify the concentrations of the unknown analytes from a calibration curve using known amounts of spiked analytes included in an endogenous-free matrix. The spiked analytes can be highly pure standards which are not isotopically enriched, or high purity isotopically enriched standards that are different in MRM transitions from the internal standard.

In some embodiments, the present teachings provide a method for quantifying ketosteroids and analytes containing keto or aldehyde functionality. In some embodiments, the method can comprise derivatization chemistry and a liquid chromatography/tandem mass spectrometry (LC/MSMS) workflow. The method can comprise using a permanently charged aminooxy reagent which can significantly increase the detection limits of ketosteroids.

These and other features of the embodiments as will be apparent are set forth and described herein.

### BRIEF DESCRIPTION OF THE DRAWINGS

A detailed description of various embodiments is provided herein below with reference, by way of example, to the following drawings. The skilled person in the art will understand that the drawings, described below, are for illustration purposes only. The drawings are not intended to limit the scope of the applicant's teachings in any way.

FIG. 1 is a flow diagram of a method showing sample preparation, derivatization, and LC/MS/MS analysis of the derivatized analyte.

FIGS. 2A and 2B are flow diagrams of two method showing sample preparation, derivatization, and LC/MS/MS analysis of the derivatized analyte. Testosterone used as an example for both FIG. 2A and FIG. 2B.

FIGS. 3A and 3B show concentration curves from 0 to 5000 pg/mL of testosterone. FIG. 3A provides the concentration curve spiked in Double Charcoal Stripped (DCS) human serum using a fast chromatographic gradient method which co-elutes the two positional isomers which are formed after derivatization. FIG. 3B provides the concentration curve spiked in DCS human serum, using a shallower chromatographic gradient which separates the E/Z isomers which are formed after derivatization. The integration is the sum of the areas of both isomers peaks.

FIG. 4 shows the MS/MS fragments and spectrum of QAO Testosterone using CE=62 eV at which the signature ions contain fragments from both testosterone structure and from the derivatizing reagent structure, according to various embodiments of the present teachings.

FIGS. 5A and 5B show the chromatograms of QAO derivatized testosterone using MRM transition of a targeted Q3 fragment (FIG. 5A) as compared to neutral loss Q3 fragment (FIG. 5B), according to various embodiments of the present teachings using API 4000™ LC/MSMS

FIG. 6 shows the targeted MS/MS fragmentation and spectrum of QAO Progesterone and the MS/MS spectrum of QAO Progesterone at CE=45 eVAPI 4000™ LC/MSMS

FIGS. 7A-7C show the LC/MS/MS chromatograms of progesterone at CE=45 eV and illustrates a background noise reduction in an LC/MS/MS analysis using API 4000™ LC/MSMS

FIGS. 8A and 8B show representative chromatograms of Testosterone analysis in human serum, API 3200™ LC/MSMS. FIG. 8A is 10 pg/mL standard of testosterone (Te) spiked in a stripped serum extracted by SLE and derivatized with QAO reagent. FIG. 8B is a sample from a female pediatric patient, age 11 (approximately 10 pg/mL extracted by SLE and derivatized)

FIG. 9 provides a Testosterone Concentration curve 10-10000 pg/mL (200 μL serum, increasing spiked concentrations of d₃Te and 500 pg/mL ¹³C Te Internal Standard (IS)). Dynamic range covers the reference values of all human samples using API 3200™ LC/MSMS system.

FIG. 10 shows a DBS concentration curve 50-1000 pg/mL using d₃Te as calibrant and ¹³C Te as IS, 10 μL of female whole blood spiked on filter paper disc of ¼″ diameter.

FIGS. 11A-11C shows the chromatogram of a QAO derivatized female dried blood spot, 10 μL whole blood. (QTRAP® 5500 system). The measurement of its endogenous Te concentration is ˜43 pg/mL. FIG. 11A is a chromatogram of ¹³C Te as internal standard 500 pg/mL. FIG. 11B is a chromatogram of 50 pg/mL spiked d₃Te. FIG. 11C is a chromatogram of Endogenous d₀Te in the sample. The concentration was measured as approximately 43 pg/mL.

FIGS. 12A-12B show chromatograms of underivatized DBS from 10 μL female whole blood (same donor presented in FIGS. 11A-11C), using AB SCIEX QTRAP® 5500 System. FIG. 12A is a chromatogram of a d3 Te internal standard. FIG. 12B is a chromatogram of 10 μL female whole blood. No signal of underivatized Te could be detected.

FIGS. 13A-13E. FIGS. 13A-13D are chromatograms analyzing free testosterone in female serum (pool) using QAO derivatization and QTRAP® 5500 Instrument. FIG. 13A shows ¹³C Te used as internal standard while FIG. 13B shows total Te with 200 μL serum. d₃Te is spiked as calibrant in the concentration curve. FIGS. 13C and 13D show the IS and free Te after Ultra Filtration of 30 KD Molecular Weight cutoff membrane. The free Te concentration is estimated as 0.94 pg/mL which is 1.13% of the total Testosterone concentration. FIG. 13E is a concentration curve showing the lower limit of quantitation of free testosterone for 200 μL ultra filtrate (UF) is ˜1 pg/mL.

FIGS. 14A and 14B are chromatograms showing an estimate of Free Testosterone concentration in female saliva (1 mL) using QAO derivatization and QTRAP® 5500 Instrument. 20 pg/mL d₃Te used as IS. FIG. 14A shows an endogenous free testosterone estimated as 2.1 pg/mL and FIG. 14B shows a 20 pg/mL d₃ testosterone internal standard.

FIG. 15 are representative LC/MS/MS Chromatograms of isobaric ketosteroids.

It will be understood that the drawings are exemplary only and that all reference to the drawings is made for the purpose of illustration only, and is not intended to limit the scope of the embodiments described herein below in any way. For convenience, reference numerals may also be repeated (with or without an offset) throughout the figures to indicate analogous components or features.

### DETAILED DESCRIPTION OF VARIOUS EMBODIMENTS

It will be appreciated that for clarity, the following discussion will explicate various aspects of embodiments of the teachings, but omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments may not require certain of the specifically described details in every implementation, which are set forth herein only to provide a thorough understanding of the embodiments. Similarly it will be apparent that the described embodiments may be susceptible to slight alteration or variation according to common general knowledge without departing from the scope of the disclosure. The following detailed description of embodiments is not to be regarded as limiting the scope of the present teachings in any manner.

The ketone and aldehyde compounds used as analytes in the mass spectrometry techniques described herein are found in a variety of biological matrices such as physiological fluid samples, cell or tissue lysate samples, protein samples, cell culture samples, fermentation broth media samples, agricultural product samples, animal product samples, animal feed samples, samples of food or beverage for human consumption, combinations thereof, and the like, and essentially any sample where the ketone and aldehyde functionality is present in the analyte. Examples of biological matrices comprise the physiological fluids, such as blood, serum, plasma, sweat, tears, urine, peritoneal fluid, lymph, vaginal secretion, semen, spinal fluid, ascetic fluid, saliva, sputum, breast exudates, and combinations thereof. In some embodiments, the samples are from a dried blood spot (DBS).

To demonstrate the applicability of the present techniques to ketone and aldehyde compounds, ketosteroids are analyzed and measured in the Examples below. The quantitation of ketosteroids present a particular challenge due to their low concentrations in the biological matrices of common clinical samples.

The present teachings can be applied to both natural and synthetic ketone and aldehyde analytes. Ketosteroids comprise, but are not limited to, DHT, testosterone, epitestosterone, desoxymethyltestosterone (DMT), tetrahydrogestrinone (THG), aldosterone, estrone, 4-hydroxyestrone, 2-methoxyestrone, 2-hydroxyestrone, 16-ketoestradiol, 16 alpha-hydroxyestrone, 2-hydroxyestrone-3-methylether, prednisone, prednisolone, pregnenolone, progesterone, DHEA (dehydroepiandrosterone), 17 OH pregnenolone, 17 OH progesterone, 17 OH progesterone, androsterone, epiandrosterone, and D4A (delta 4 androstenedione) can be analyzed in various embodiments of the present teachings.

The samples may be enriched by various methods. The enrichment method is dependent upon the type of sample, such as blood (fresh or dried), plasma, serum, urine, or saliva. Exemplary enrichment methods include protein precipitation, liquid-liquid extraction, solid-liquid extraction, and ultrafiltration. Other enrichment methods, or the combination of two or more enrichment methods may be used.

Labeling Reagent

Thus, there is provided herewith a method for mass analysis of a ketone or aldehyde using a specific labeling reagent and selection of a signature ion fragment for analysis.

In some embodiments, there is provided labeling reagents and sets of labeling reagents for the relative quantitation, absolute quantitation, or both, of ketone compounds and/or aldehyde compounds in biological samples including a labeling reagent having general formula (I):

Y—(CH₂)ₙ—O—NH₂ (I)

where n is 2, 3, 4, 5, or 6 and Y has the structure:

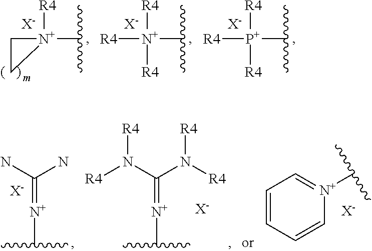

each R₄ is independently H or a C₁-C₁₈ alkyl which is branched or straight chain,

m is an integer between 1 and 20, and

X is an anion,

or a salt or a hydrate thereof.

In some embodiments, n is 2-4 and in other embodiments, n is 3. In some embodiments, Y is —N(CH₃)⁽⁺⁾. In some embodiments, m is an integer between 1 and 12 or an integer between 1 and 5. In some embodiments each R₄ is independently H or a C₁-C₁₂ alkyl which is branched or straight chain, or each R₄ is independently H or a C₁-C₆ alkyl which is branched or straight chain. In some embodiments, each R₄ is the same.

In some embodiments, the compound of formula (I) is a salt. In some embodiments, the salt is CF₃COO—; CF₃CF₂COO—; CF₃CF₂CF₂COO—; or CF₃SO₃COO—. In some embodiments, the salt is a perfluorocarboxylate salt.

In some embodiments, the labeling reagent of formula I is:

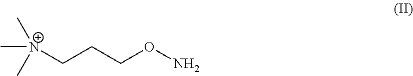

or a salt or hydrate thereof. In some embodiments, the compound of formula (II) is a salt. In some embodiments, the salt is CF₃COO—; CF₃CF₂COO—; CF₃CF₂CF₂COO—; or CF₃SO₃COO—. In some embodiments, the salt is a perfluorocarboxylate salt.

In various aspects, the present teachings provide labeled analytes, wherein the analyte can comprise at least one ketone group and the labeling reagent of formula (I) and/or (II). In various aspects, the present teachings provide labeled analytes, wherein the analyte can comprise at least one aldehyde group and the label described herein.

In various embodiments, the labeling reagents of formula (I) and/or (II) are used to label internal standards (IS). Isotopically labeled internal standards of many ketosteroids and other aldehyde compounds are not available commercially. Additionally, the standards that are available are often expensive and limited in form. For example d₃ testosterone IS can only be purchased in solution and significant deviations from the reported concentrations may be found. While ¹³C testosterone IS, if available, is more stable, both the Q1 and Q3 masses are different from the analyte.

Thus, in some embodiments, “heavy” (isotopically enriched) QAO reagent can provide internal standards for every keto-steroid. In some embodiments, these internal standards are particularly advantageous if a panel of steroids is to be analyzed. Thus, isotopically enriched analogues of the labeling regent can be used and internal standards can be generated for quantitation. For example, heavy atom isotopes of carbon (¹²C, ¹³C, and ¹⁴C), nitrogen (¹⁴N and ¹⁵N), oxygen (¹⁶O and ¹⁸O), sulfur (³²S, ³³S, and ³⁴S), and/or hydrogen (hydrogen, deuterium and tritium) can be used in the preparation of internal standards. United States Patent Application Publication No. US 2005/068446 A1 discloses synthesis of isotopically enriched compounded; mass analysis workflows and strategies are disclosed in U.S. Patent Application Publication No. US 2008/0014642 A1, both of which are incorporated herein in their entireties by reference.

The isotopically enriched compounds may comprise, for example:

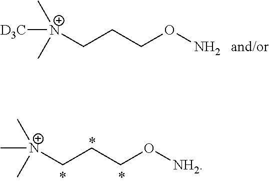

The method can involve using an MRM workflow for quantitative analysis of ketosteroids. The reagents can be isotope-coded for quantitative analysis of an individual or of a panel of keto compounds. In studies involved ketosteroid profiling the MS/MS fragmentation can be targeted at low collision energies to produce predominantly the neutral loss signature ion from the aminooxy-derivatized product. The MRM transition can be the mass of the derivatized steroid in Q1 and the mass of the neutral loss fragment in Q3. The present teachings provide, in some embodiments, a process for significantly reducing background noise via derivatization, resulting in improved sensitivity and targeted selection of Q3 fragments resulting in improved specificity.

Thus, there is provided herewith, in some embodiments, a set of isotopically labeled internal standards of steroids such as testosterone. This set comprises two or more adducts that comprise a known concentration of a ketosteroid labeled with the QAO reagent as described herein, wherein each of the two or more adducts have different isotopically enriched analogues.

The present teachings comprise reagents and methods using mass differential tags including sets of mass differential labels where one or more labels of the set contains one or more heavy atom isotopes. A set of mass differential labels can also be provided by preparing labels with different overall mass and different primary reporter groups or mass balance groups, although not every member of a set of mass differential tags need to be isotopically enriched. The present reagents and methods enable analysis of ketone and aldehyde analytes in one or more samples using mass differential labels and parent-daughter ion transition monitoring (PDITM). The present teachings can be used for qualitative and quantitative analysis of such analytes using mass differential tagging reagents and mass spectrometry. The mass differential tags comprise, but are not limited to, non-isobaric isotope coded reagents and the present teachings comprise reagents and methods for the absolute quantitation of ketone and aldehyde compounds with or without the use of an isotopically enriched standard compound.

Thus, provided herewith are sets of mass differential labels of general formula (I) and/or (II). In various embodiments, provided are sets of isobaric labels of general formula (I) in their unsalted and/or unhydrated form. In various embodiments, the masses of the labels differ by less than about 0.05 AMU in the unsalted and/or unhydrated form. The sets of labels provided comprise two or more compounds of the general formula (I) or (II) wherein one or more of the compounds in the set of labels contains one or more heavy atom isotopes. In various embodiments, the heavy atom isotopes are each independently ¹³C, ¹⁵N, ¹⁸O, ³³S, or ³⁴S.

The compounds of formula (I) or (II) can be provided in a wide variety of salt and hydrate forms including, but not limited to, a mono-TFA salt, a mono HCl salt, a bis-HCl salt, or a bis-TFA salt, or a hydrate thereof. Variation on formula (I) are disclosed in U.S. Pat. Publ. 2011/0003395 and WO2005/068446, both of which are specifically incorporated by reference and are generally referred to as iTRAQ reagents.

According to various embodiments, isotopes can be used as balance groups or balance moieties, for example, isotopes of hydrogen, carbon, nitrogen, oxygen, sulfur, chlorine, bromine, and the like. Exemplary balance groups or moieties can also comprise those described, for example, in U.S. Patent Application Publications Nos. US 2004/0219685 A1, published Nov. 4, 2004, US 2004/0219686 A1, published Nov. 4, 2004, US 2004/0220412 A1, published Nov. 4, 2004, and US 2010/0112708 A1, published May 6, 2010, all of which are incorporated herein in their entireties by reference.

In various embodiments, the one or more of the compounds of the set of labels is isotopically enriched with two or more heavy atoms; three or more heavy atoms; and/or with four or more heavy atoms. In various embodiments, a set of labels of formula incorporated heavy atom isotope such that the isotopes are present in at least 80 percent isotopic purity, at least 93 percent isotopic purity, and/or at least 96 percent isotopic purity.

Alternatively or in addition to mass differential tags, isobaric tags can be used. When isotopically enriched isobaric tags are used, sets of isobaric labels may comprise one, or more heavy atom isotopes. A set of isobaric labels can have an identical or specifically defined range of aggregate masses but has a primary reporter ion or charged analyte of a different measurable mass. A set of isobaric reagents enables both qualitative and quantitative analysis of ketone and aldehyde analyte compounds using mass spectroscopy. For example, isotopically enriched isobaric tags and parent-daughter ion transition monitoring (PDITM) can measure or detect one or more ketone or aldehyde compounds in a sample such as a specific ketosteroid or group of ketosteroids.

In embodiments comprising sets of isobaric labels, the linker group portion can be referred to as a balance group. For example, a set of four isobaric labels are added to a set of one or more analytes and combined to form a combined sample that is subjected to MS/MS analysis to fragment the labeled ketone or aldehyde compound and produce 4 reporter ions of different mass or charged analytes. The labels can be made isobaric by an appropriate combination of heavy atom substitutions of a reporter group or mass balance group or portion thereof or a mass balance group alone or portion thereof.

Analysis

The present teachings can provide reagents and methods for the analysis of one or more ketone or aldehyde compound in one or more samples using mass differential labels, isobaric labels, or both, and parent-daughter ion transition monitoring (PDITM). The present teachings can provide methods for determining the relative concentration, absolute concentration, or both, of one or more analytes in one or more samples and provide methods whereby the relative concentration, absolute concentration, or both, of multiple analytes in a sample, one or more analytes in multiple samples, or combinations thereof, can be determined in a multiplex fashion using mass differential tagging reagents, isobaric tagging reagents, or both, and mass spectroscopy.

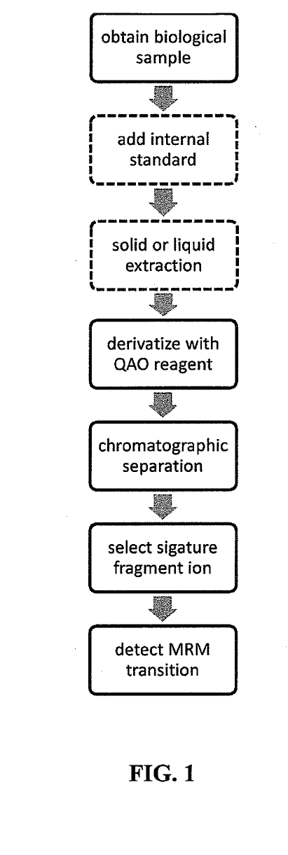

Thus, some of the various methods as described herein can be explained by the flow diagram of FIG. 1. Particularly, a sample, which may be part a biological matrix such as blood, serum, plasma, urine, or saliva can be selected and derivatized with the labeling reagent such as QAO by aminooxy chemistry followed by the mixing the labeled analyte and a QAO labeled standard. The mixture can be subjected to chromatographic separation, for example, by LC such as by HPLC, followed by mass analysis by MRM. If isotopically enriched reagent is used as an internal standard, it is preferably added after the derivatization step.

The signature fragment ion measured in the MRM can be carefully selected to comprise structural fragments with attached labeling reagent or part thereof. For example, where the labeling reagent includes a trimethyl amine, the signature fragment ion may include an ion which has lost the moiety N(CH₃) as well as at least a portion of the backbone of the analyte. In some embodiments, the collision energy selected to form the signature fragment ion is a low collision energy so as to produce a single predominant signature fragment ion. In some embodiments, the collision fragment energy is selected to be 65, (for example, in a range of 30 to 130 ev).

With judicious selection of the signature ion fragment which comprises at least part of the readily ionizable labeling reagent, mass analysis can be done with significantly lower background noise compared to MRM analysis of the ketone or aldehyde species analyzed without the addition of the labeling reagent. For example, in some embodiments, the background noise is reduced to provide a lower limit of quantitation of 100 pg/mL or less, or 50 pg/mL or less, or 10 pg/mL or less, where the sample was obtained from a biological matrix.

In some embodiments, the signature ion fragment is a neutral loss fragment that contains a portion of the analyte backbone and also contains a portion of the labeling reagent. In some embodiments, the signature ion fragment, when the ketone being analyzed is a testosterone or testosterone derivative, is one or more fragment having a m/z of 164.2 and 152.3. In these embodiments and in other embodiments, the signature ion fragment can be isotopically enriched, such as a ¹³C enriched fragment having a m/z of 167.2 and/or 155.2.

Quantitation can be enabled by relative or absolute measurement of the signal derived from one or more analytes and standards. The positive charge can be transferred to the analyte which functions as the fragment ion to be detected by mass spectrometry.

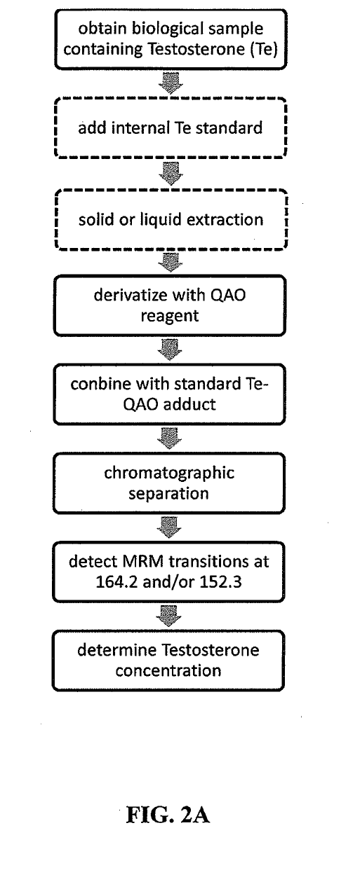

Other various methods as described herein can be explained by the flow diagrams of FIG. 2A and FIG. 2B. Particularly, a sample containing testosterone or a derivative thereof, which is part a biological matrix such as blood, serum, plasma, urine, or saliva, is selected. An internal testosterone standard, such as d₃ testosterone is optionally added. The testosterone analyte can then be optionally extracted by, for example, liquid/liquid extraction or solid/liquid extraction. The sample and optionally the internal standard are derivatized with the QAO labeling reagent by aminooxy chemistry. In the method described in FIG. 2A, the labeled adduct is combined with a testosterone standard that has also been labeled with a QAO labeling reagent. For the methods described in FIG. 2A and FIG. 2B, the mixture is subjected to chromatographic separation, for example, by LC such as by HPLC, followed by mass analysis by MRM where the MRM transitions are 164.2 and/or 152.3 are analyzed. Quantitation is enabled by relative or absolute measurement of the signal derived from one or more analytes and standards. In FIG. 2B, the Te concentration is determined based on a concentration curve. The positive charge is transferred to the analyte which functions as the fragment ion to be detected by mass spectrometry.

Quantitation can be enabled by spiking increased amounts of known analytes concentrations into an endogenous-free matrix to create a calibration curve. The unknown concentrations of the samples are calculated from the linear regression of the concentration curve. The linear plot of the concentration curve comprises of the concentration ratios of the calibrants and the internal standard versus the area ratios of the calibrants and the IS. Alternatively, relative quantitation can be enabled by a one point calibration using the known amounts of the spiked internal standards. For samples which are isobaric isomers, the chromatographic separation can be used to separate the samples prior to their mass analysis since these compounds may have the same mass patterns. Since isobaric ketosteroid in the biological sample may have a similar Q1/Q3 MRM transition, the isobaric ketosteroids can share the same fragmentation pattern with the analyte in order to appear as interference. In such a scenario, the isobaric ketosteroid is preferably chromatographically separated from the analyte.

An added advantage of the labeling reagent is that, in some embodiments, upon MSMS fragmentation, the derivatized analyte generates a fragment ion (Q3 signature ion) with the charge on the derivatized analyte which makes it amenable to MS3 analysis.

The derivatized analyte can enhance both the sensitivity and selectivity in the mass spectrometer. For example, the presently claimed methods may be used to detect testosterone from a biological matrix with a sensitivity that is 40-50 times that of an underivatized sample. In some embodiments, the MS/MS sensitivity is enhanced 20 fold, 50 fold, 100 fold, 500 fold, or even 1000 fold depending on the compound. In some embodiments, the limit of detection after derivatization can be as low as <1 pg/mL.

In various embodiments, the step of adding a label to the standard sample to label one or more of the standard compounds in the sample comprises a one step reaction where the aminooxy group forms an oxime with the ketone or aldehyde group of the analyte standard.

In various aspects, the present teachings provide methods for labeling a ketoanalyte to form a labeled analyte compound. In various embodiments, the methods comprise reacting a labeling compound of the general formula (I) or (II) with a ketone-containing compound. Specifically, exemplary ketosteroids were derivatized with the labeling reagent of formula I and specifically labeled in 10% acetic acid in MeOH for 30 minutes at room temperature or 60 minutes at 60 C for bis ketosteroids.

The present teachings can be applied to both naturally produced as well as synthetic ketosteroids. Examples of ketosteroids, including, but not limited to, any steroid, metabolite or derivation thereof containing a ketone moiety, such as the keto-forms of cortisol, 11-desoxycortisol (compound S), corticosterone, DHT, testosterone, epitestosterone, desoxymethyltestosterone (DMT), tetrahydrogestrinone (THG), estrone, 4-hydroxyestrone, 2-methoxyestrone, 2-hydroxyestrone, 16-ketoestradiol, 16 alpha-hydroxyestrone, 2-hydroxyestrone-3-methylether, prednisone, prednisolone, pregnenolone, progesterone, DHEA (dehydroepiandrosterone), 17 OH pregnenolone, 17 OH progesterone, 17 OH progesterone, androsterone, epiandrosterone, D4A (delta 4 androstenedione), 21 deoxycortisol, 11 deoxycorticosterone, Allopregnanolone, and Aldosterone.

Referring to the Examples, Figures, and Tables below, an example of labeling ketosteroid analytes such as testosterone, aldosterene, pregnenolone, and progesterone, with labeling reagents is shown. In these reactions, the aminooxy moiety reacts with the ketone or aldehyde on the steroid to form an oxime group on the labeled compound to yield a labeled analyte.

As described herein, methods for determining the concentration of one or more ketone or aldehyde compounds in two or more samples are provided by adding a different label to each sample, combining the differentially labeled samples and using PDITM to determine a concentration of one or more of the analyte compounds in the samples. One of the samples may comprise a standard sample, such as a control sample, a reference sample, a sample with a compound of known concentration, etc. The methods can thus provide an analysis of multiple compounds from multiple samples.

In various embodiments, the step of determining the concentration of one or more labeled ketone or aldehyde analyte compounds comprises determining the absolute concentration of one or more of the labeled ketone or aldehyde analyte compounds, determining the relative concentration of one or more of the labeled ketone or analyte compounds, or combinations of both.

Certain methods comprise the steps of labeling one or more ketone or aldehyde compounds, in two or more samples of interest by adding to each sample of interest a different tag from a set of tags to form a panel of labeled ketone or aldehyde analyte compounds. Each tag from the set of tags may comprise the labeling reagent or portion thereof as described herein. One or more of the labeled ketone or aldehyde analyte compounds may be differentially labeled with respect to the sample from which each analyte was obtained or in which it is contained. The step of adding a label to a ketone or aldehyde compound may comprise a one step reaction where a first portion of the label is comprised of the formula (I) or (II).

A portion of each of the samples can be combined to produce a combined sample and a portion thereof analyzed by parent-daughter ion transition monitoring and measuring the ion signal of one or more of the transmitted ions. The transmitted parent ion m/z range can comprise an m/z value of the labeled analyte compound and the transmitted daughter ion m/z range comprises an m/z value of a reporter ion derived to the tag of the labeled analyte compound or is the ionized analyte itself. The concentration of one or more of the labeled analyte compounds can then be determined based at least on a comparison of the measured ion signal of the corresponding transmitter reporter or analyte ions to one or more measured ion signals of a standard compound. The ion signal(s) can, for example, be based on the intensity (average, mean, maximum, etc.) of the ion peak, an area of the ion peak, or a combination thereof. One or more of the two or more samples of interest can be a standard sample containing one or more the standard compounds.

In some embodiments, the concentration of a ketone or aldehyde compound is determined by comparing the measured ion signal of the corresponding labeled aldehyde ketone analyte compound-reporter ion transition signal to one or more of:
(i) a concentration curve for a standard compound-reporter or analyte ion transition; or
(ii) a standard compound-reporter ion transition signal for a standard compound in the combined sample with the labeled ketone or aldehyde analyte compound.

In some embodiments, the “Parent-daughter ion transition monitoring” or “PDITM” is used as the method of analysis and workflow status. PDITM refers to a technique whereby the transmitted mass-to-charge (m/z) range of a first mass separator (often referred to as “MS” or the first dimension of mass spectrometry) is specifically selected to transmit a molecular ion (often referred to as “the parent ion” or “the precursor ion”) to an ion fragmentor (e.g. a collision cell, photodissociation region, etc.) to produce fragment ions (often referred to as “daughter ions”) and the transmitted m/z range of a second mass separator (often referred to as “MS/MS” or the second dimension of mass spectrometry) is selected to transmit one or more daughter ions to a detector which measures the daughter ion signal. This technique offers unique advantages when the detection of daughter ions in the spectrum is focused by “parking” the detector on the expected daughter ion mass. The combination of parent ion and daughter ion masses monitored can be referred to as the “parent-daughter ion transition” monitored. The daughter ion signal at the detector for a given parent ion-daughter ion combination monitored can be referred to as the “parent-daughter ion transition signal”.

For example, one embodiment of parent-daughter ion transition monitoring is multiple reaction monitoring (MRM) (also referred to as selective reaction monitoring). In various embodiments of MRM, the monitoring of a given parent-daughter ion transition comprises using as the first mass separator (e.g., a first quadrupole parked on the parent ion m/z of interest) to transmit the parent ion of interest and using the second mass separator (e.g., a second quadrupole parked on the daughter ion m/z of interest) to transmit one or more daughter ions of interest. In various embodiments, a PDITM can be performed by using the first mass separator (e.g., a quadrupole parked on a parent ion m/z of interest) to transmit parent ions and scanning the second mass separator over a m/z range including the m/z value of the one or more daughter ions of interest.

For example, a tandem mass spectrometer (MS/MS) instrument or, more generally, a multidimensional mass spectrometer instrument, can be used to perform PDITM, e.g., MRM. Examples of suitable mass analyzer systems comprise, but are not limited to, those that comprise one or more of a triple quadrupole, a quadrupole-linear ion trap, a quadrupole TOF, and a TOF-TOF.

Thus, PDITM can be performed on a mass analyzer system comprising a first mass separator, and ion fragmentor and a second mass separator. The transmitted parent ion m/z range of a PDITM scan (selected by the first mass separator) is selected to comprise a m/z value of one or more of the labeled analyte compounds and the transmitted daughter ion m/z range of a PDITM scan (selected by the second mass separator) is selected to comprise a m/z value of one or more of the reporter ions corresponding to the tag of the transmitted labeled analyte compound.

In some embodiments, parent daughter ion transition monitoring (PDITM) of the labeled analytes is performed using a triple quadrupole MS platform. More details about PDITM and its use are described in U.S. Patent Application Publication No. US 2006/0183238 A1, which is incorporated herein in its entirety by reference. In some embodiments, the aminooxy MS tagging reagent undergoes neutral loss during MSMS and leaves a reporter ion that is a charged analyte species. In some embodiments, the aminooxy MS tagging reagent forms a reporter ion that is a tag fragment during MSMS.

Thus, in various embodiments, for analyzing one or more ketone or aldehyde analyte compounds in one or more samples using labels of the present teachings comprises the steps of: (a) labeling one or more analyte compounds each with a different label from a set of labels of formula (II) providing labeled analyte compounds, the labeled analyte compounds each having a mass balance or reporter ion portion; (b) combining at least a portion of each of the labeled analyte compounds to produce a combined sample; (c) subjecting at least a portion of the combined sample to parent-daughter ion transition monitoring; (d) measuring the ion signal of one or more of the transmitted analyte or reporter ions; and (e) determining the concentration of one or more of the labeled ketone or aldehyde analyte compounds based at least on a comparison of the measured ion signal of the corresponding analyte or reporter ion to one or more measured ion signals of a standard compound. Accordingly, in various embodiments, the concentration of multiple analyte compounds in one or more samples can be determined in a multiplex fashion, for example, by combining two or more labeled analyte compounds to produce a combined sample and subjecting the combined sample to PDITM, and monitoring the analyte or reporter ions of two or more of labeled analyte compounds.

The tags added to the two or more samples are selected from a set of tags within one experimental measurement: (i) multiple aldehyde or ketone analyte compounds from different samples (e.g., a control, treated, time sequence of samples) can be compared and/or quantified; (ii) multiple concentration measurements can be determined on the same ketone or aldehyde compound from different samples; and (iii) different isolates of a clinical sample can be evaluated against a baseline sample; etc.

The step of subjecting at least a portion of the combined sample to PDITM comprises loading the portion of the combined sample on a chromatographic column (e.g., a LC column, a gas chromatography (GC) column, or combinations thereof), subjecting at least a portion of the eluent from the chromatographic column to parent-daughter ion transition monitoring and measuring the ion signal of one or more of the transmitted reporter ions.

The chromatographic column is used to separate two or more labeled analyte compounds, which differ in the analyte portion of the labeled compound. For example, a first labeled aldehyde or ketone compound found in one or more of the samples is separated by the chromatographic column from a second labeled ketone analyte compound found in one or more of the samples. Two or more different labeled analyte compounds are separated such that the different compounds do not substantially co-elute. Such chromatographic separation can further facilitate the analysis of multiple compounds in multiple samples by, for example, providing chromatographic retention time information on a compound.

The one or more measured ion signals of a standard compound used in the step of determining the concentration of one or more of the labeled analyte compounds can be provided in many ways. In various embodiments, one or more non-isotopically enriched standard compounds are labeled with a tag and at least a portion of one or more of the one or more labeled standard compounds is combined with at least a portion of each of the labeled analyte compounds to produce a combined sample; followed by subjecting at least a portion of this combined sample to PDITM and measuring the ion signal of one or more of the transmitted reporter ions.

A tag from the set of tags is added to one or more standard samples to provide one or more labeled standard samples, each standard sample containing one or more non-isotopically enriched standard compounds that are labeled by the tag, the tag added to the one or more standard samples being different from the tags added to the samples of interest. At least a portion of one or more of the one or more labeled standard samples is combined with at least a portion of each of the samples of interest to produce a combined sample; followed by subjecting at least a portion of this combined sample to PDITM and measuring the ion signal of one or more of the transmitted reporter ions.

The measured ion signals of one or more of the reporters or analyte ions corresponding to one or more of the one or more labeled standard compounds in the combined sample can then be used in determining the concentration of one or more of the labeled analyte compounds and can be used to generate a concentration curve by plotting several values for standard compounds. Accordingly, determining the concentration of a labeled analyte compound is based at least on a comparison of the measured ion signal of the corresponding reporter or analyte ions to the measured ion signal of one or more reporter or analyte ions corresponding to one or more of the one or more labeled standard compounds in the combined sample. The step of subjecting at least a portion of this combined sample to PDITM can comprise, e.g., a direct introduction into a mass analyzer system; first loading at least a portion of this combined sample on a chromatographic column followed by subjecting at least a portion of the eluent from the chromatographic column to PDITM and measuring the ion signal of one or more of the transmitted reporter ions.

As disclosed herein, PDITM on a standard compound can be performed on a mass analyzer system comprising a first mass separator, and ion fragmentor and a second mass separator. The transmitted parent ion m/z range of a PDITM scan (selected by the first mass separator) is selected to comprise a m/z value of one or more of the labeled standard compounds and the transmitted daughter ion m/z range of a PDITM scan (selected by the second mass separator) is selected to comprise a m/z value one or more of the reporter or analyte ions corresponding to the transmitted standard compound.

Determining the concentration of one or more of the labeled analyte compounds can be based on both: (i) a comparison of the measured ion signal of the corresponding reporter or analyte ion to the measured ion signal of one or more reporter or analyte ions corresponding to one or more concentration curves of one or more standard compounds, and (ii) a comparison of the measured ion signal of the corresponding reporter ion to the measured ion signal of one or more reporter ions corresponding to one or more labeled standard compounds combined with the labeled ketone or aldehyde analyte. A non-isotopically enriched standard compound is provided having a first concentration and labeled with a tag from the set of tags is combined with at least a portion of each of the labeled samples to produce a combined sample, and this combined sample can then be further analyzed as described herein.

Thus, in various embodiments of the present teachings, a concentration curve of a standard compound can be generated by: (a) providing a isotopically or non-isotopically enriched standard ketone or aldehyde compound having a first concentration; (b) labeling the standard compound with a label from a set of labels wherein the labeled ketone standard compound has a reporter ion portion; (c) loading at least a portion of the labeled standard compound on a chromatographic column; (d) subjecting at least a portion of the eluent from the chromatographic column to parent-daughter ion transition monitoring; (e) measuring the ion signal of the transmitted analyte or reporter ions; (f) repeating steps (a)-(e) for one or more different standard compound concentrations; and (g) generating a concentration curve for the standard compound based at least on the measured ion signal of the transmitted analyte or reporter ions at one or more standard compound concentrations.

The present disclosure provides methods for determining the concentration of one or more ketone or aldehyde analyte compounds in one or more samples. The methods comprise the steps of labeling one or more ketone or aldehyde compounds each with a different tag from a set of tags of formula (I), where the Y group, which may be a quaternary nitrogen from each tag from the set of tags comprises a reporter ion portion, at least a portion of each of the labeled analyte compound can be combined to produce a combined sample and at least a portion of the combined sample can be subjected to parent-daughter ion transition monitoring (where the transmitted parent ion m/z range comprises a m/z value of the labeled analyte compound and the transmitted daughter ion m/z range comprises a m/z value of a reporter ion corresponding to the tag of the labeled analyte compound) and measuring the ion signal of one or more of the transmitted reporter ions; then determining the concentration of one or more of the labeled analyte compounds based at least on a comparison of the measured ion signal of the corresponding reporter ion to one or more measured ion signals of a standard compound. The ion signal(s) can, for example, be based on the intensity (average, mean, maximum, etc.) of the ion peak, an area of the ion peak, or a combination thereof.

PDITM can be performed on any suitable mass analyzer known in the art, including a mass analyzer system comprising a first mass separator, and ion fragmentor and a second mass separator. The transmitted parent ion m/z range of a PDITM scan (selected by the first mass separator) is selected to comprise a m/z value of one or more of the labeled analyte compounds and the transmitted daughter ion m/z range of a PDITM scan (selected by the second mass separator) is selected to comprise a m/z value one or more of the reporter ions corresponding to the tag of the transmitted labeled analyte compound.

The one or more ketone or aldehyde compound samples are labeled with one or more of tags selected from a set of mass differential tags so that within the same experimental measurement: (i) multiple ketone or aldehyde containing compounds from different samples (e.g., a control, treated) can be compared and/or quantified; (ii) multiple concentration measurements can be determined on the same ketone or aldehyde compound from the same sample; and (iii) different isolates of a clinical sample can be evaluated against a baseline sample.

The step of subjecting at least a portion of the combined sample to PDITM comprises introducing the combined sample directly into a mass analyzer system, e.g., by introduction of the combined sample in a suitable solution using an electrospray ionization (ESI) ion source.

The measured ion signals of one or more of the reporters ions corresponding to one or more of the one or more labeled standard compounds in the combined sample determines the concentration of one or more of the labeled analyte compounds. Determining the concentration of a labeled analyte compound is based at least on a comparison of the measured ion signal of the corresponding fragment ion to the measured ion signal of one or more fragment ion corresponding to one or more of the one or more labeled standard compounds in the combined sample. The step of subjecting at least a portion of this combined sample to PDITM can comprise, e.g., a direct introduction into a mass analyzer system; first loading at least a portion of this combined sample on a chromatographic column followed by subjecting at least a portion of the eluent from the chromatographic column to PDITM and measuring the ion signal of one or more of the transmitted reporter or analyte ions; or combinations thereof.

In some embodiments, determining the concentration of one or more of the labeled analyte compounds comprises a comparison of the measured ion signal of the corresponding analyte or reporter ion to the measured ion signal of one or more reporter ions corresponding to one or more concentration curves of one or more standard compounds. A non-isotopically enriched standard compound is provided having a first concentration and labeled with a tag from a set of tags. A portion of the labeled standard compound is subjected to parent-daughter ion transition monitoring (where the transmitted parent ion m/z range comprises a m/z value of the labeled standard compound and the transmitted daughter ion m/z range comprises a m/z value of a reporter or analyte ion corresponding to the tag of the labeled standard compound) and the ion signal of the reporter or analyte ion is measured. The steps of labeling and the steps of PDITM and measuring the ion signal of the transmitted reporter or analyte ions are repeated for at least one more standard compound concentration different from the first concentration to generate a concentration curve for the standard compound.

In some embodiments, a kit including one or more of the aminooxy reagents described herein can be provided, for example, comprising one or more permanently charged aminooxy compounds of formula (I) or (II).

In some embodiments, the method can comprise using an MRM workflow for quantitative analysis of ketosteroids. The reagents can be isotope-coded for quantitative analysis of an individual or of a panel of keto compounds. For profiling studies with the MS/MS fragmentation at low collision energies can result in one predominant signature ion. The signature ion can result from a neutral loss from the aminooxy derivatized product. The MRM transition can be the mass of the derivatized steroid in Q1 and the mass of the neutral loss fragment in Q3. For low concentrations quantitation, the MS/MS fragmentation at higher collision energy that include the labeling reagent and part of the backbone of the molecule can provide a process for significantly reducing background noise via derivatization, resulting in improved sensitivity and targeted selection of Q3 fragments resulting in improved specificity.

According to various embodiments, the present teachings provide a method that reduces or eliminates background noise without the problems associated with multistep cleanup of a biological sample and chromatographic separation. In some embodiments, the method eliminates background noise by utilizing a derivatization chemistry of ketosteroids with permanently charged aminooxy reagents (QAO) and targeted fragmentation that comprises both the reagent and the backbone of the derivatized steroid. The derivatization with a readily ionized/ionizable molecule results in better ionization efficiency in ESI MS/MS which increases sensitivity to the analyte. When the fragment ion that is the Q3 signature ion is selected to comprise structural fragments with an attached derivatization reagent, or a part of the reagent, both the sensitivity and selectivity can be enhanced. The chances that a compound with exactly the same Q1/Q3 transition would be detected and create background noise interference are very low. The only possibility for a similar Q1/Q3 MRM transition would be the existence of an isobaric ketosteroid in the biological sample. The isobaric ketosteroid would have to share the same fragmentation pattern with the analyte in order to appear as interference. In such a rare scenario, the isobaric ketosteroid can be chromatographically separated from the analyte.

According to various embodiments, an added advantage of the reagent design is that on MS/MS fragmentation the reagent generates a fragment ion, that is, a Q3 signature ion, with a charge on the derivatized analyte, making it amenable to MS3 analysis. In some embodiments, the method can be implemented on classes of molecules with keto- or aldehyde functionality, the detection of which can benefit from derivatization for ultra high sensitivity analysis by MS/MS.

In some embodiments, after detecting the analyte, the relative concentration of the analyte is measured as compared to a standard compound or a standard concentration curve. In some embodiments, the absolute concentration of at least one analyte is determined. In some embodiments, a calibrant comprising a standard labeled with at least one heavy atom is used. In some embodiments, the calibrant is a compound having at least two deuterium atoms.

The present teachings provide a highly sensitive and specific analysis of ketosteroids and classes of molecules containing a keto functionality. The present teachings provide higher signal to noise ratios with very low background noise in MS/MS due to, for example, careful deletion of signature ions to include part of the labeling reagent and part of the backbone of the molecule.

Mass Analyzers

A wide variety of mass analyzer systems can be used in the present teachings to perform PDITM. Suitable mass analyzer systems comprise two mass separators with an ion fragmentor disposed in the ion flight path between the two mass separators. Examples of suitable mass separators include, but are not limited to, quadrupoles, RF multipoles, ion traps, time-of-flight (TOF), and TOF in conjunction with a timed ion selector. Suitable ion fragmentors include, but are not limited to, those operating on the principles of: collision induced dissociation (CID, also referred to as collisionally assisted dissociation (CAD)), photoinduced dissociation (PID), surface induced dissociation (SID), post source decay, by interaction with an electron beam (e.g., electron induced dissociation (EID), electron capture dissociation (ECD)), interaction with thermal radiation (e.g., thermal/black body infrared radiative dissociation (BIRD)), post source decay, or combinations thereof.

Examples of suitable mass spectrometry systems for the mass analyzer include, but are not limited to, those which comprise one or more of a triple quadrupole, a quadrupole-linear ion trap (e.g., 4000 Q TRAP® LC/MS/MS System, Q TRAP® LC/MS/MS System), a quadrupole TOF (e.g., QSTAR® LC/MS/MS System), and a TOF-TOF.

In various embodiments, the mass analyzer system comprises a MALDI ion source. In various embodiments, at least a portion of the combined sample is mixed with a MALDI matrix material and subjected to parent-daughter ion transition monitoring using a mass analyzer with a MALDI ionization source. In various embodiments, at least a portion of the combined sample loaded on chromatographic column and at least a portion of the eluent mixed with a MALDI matrix material and subjected to parent-daughter ion transition monitoring using a mass analyzer with a MALDI ionization source.

The mass spectrometer system can comprise a triple quadrupole mass spectrometer for selecting a parent ion and detecting fragment daughter ions thereof. In this embodiment, the first quadrupole selects the parent ion. The second quadrupole is maintained at a sufficiently high pressure and voltage so that multiple low energy collisions occur causing some of the parent ions to fragment. The third quadrupole is selected to transmit the selected daughter ion to a detector. In various embodiments, a triple quadrupole mass spectrometer can comprise an ion trap disposed between the ion source and the triple quadrupoles. The ion trap can be set to collect ions (e.g., all ions, ions with specific m/z ranges, etc.) and after a full time, transmit the selected ions to the first quadrupole by pulsing an end electrode to permit the selected ions to exit the ion trap. Desired fill times can be determined, e.g., based on the number of ions, charge density within the ion trap, the time between elution of different signature peptides, duty cycle, decay rates of excited state species or multiply charged ions, or combinations thereof.

One or more of the quadrupoles in a triple quadrupole mass spectrometer can be configurable as a linear ion trap (e.g., by the addition of end electrodes to provide a substantially elongate cylindrical trapping volume within the quadrupole). In various embodiments, the first quadrupole selects the parent ion. The second quadrupole is maintained at a sufficiently high collision gas pressure and voltage so that multiple low energy collisions occur causing some of the parent ions to fragment. The third quadrupole is selected to trap fragment ions and, after a fill time, transmit the selected daughter ion to a detector by pulsing an end electrode to permit the selected daughter ion to exit the ion trap. Desired fill times can be determined, e.g., based on the number of fragment ions, charge density within the ion trap, the time between elution of different signature peptides, duty cycle, decay rates of excited state species or multiply charged ions, or combinations thereof.

The mass spectrometer system can comprise two quadrupole mass separators and a TOF mass spectrometer for selecting a parent ion and detecting fragment daughter ions thereof. In various embodiments, the first quadrupole selects the parent ion. The second quadrupole is maintained at a sufficiently high pressure and voltage so that multiple low energy collisions occur causing some of the ions to fragment, and the TOF mass spectrometer selects the daughter ions for detection, e.g., by monitoring the ions across a mass range which encompasses the daughter ions of interest and extracted ion chromatograms generated, by deflecting ions that appear outside of the time window of the selected daughter ions away from the detector, by time gating the detector to the arrival time window of the selected daughter ions, or combinations thereof.

The mass spectrometer system can comprise two TOF mass analyzers and an ion fragmentor (such as, for example, CID or SID). In various embodiments, the first TOF selects the parent ion (e.g., by deflecting ions that appear outside the time window of the selected parent ions away from the fragmentor) for introduction in the ion fragmentor and the second TOF mass spectrometer selects the daughter ions for detection, e.g., by monitoring the ions across a mass range which encompasses the daughter ions of interest and extracted ion chromatograms generated, by deflecting ions that appear outside of the time window of the selected daughter ions away from the detector, by time gating the detector to the arrival time window of the selected daughter ions, or combinations thereof. The TOF analyzers can be linear or reflecting analyzers.

The mass spectrometer system can comprise a tandem MS-MS instrument comprising a first field-free drift region having a timed ion selector to select a parent ion of interest, a fragmentation chamber (or ion fragmentor) to produce daughter ions, and a mass separator to transmit selected daughter ions for detection. In various embodiments, the timed ion selector comprises a pulsed ion deflector. In various embodiments, the ion deflector can be used as a pulsed ion deflector. The mass separator can comprise an ion reflector. In various embodiments, the fragmentation chamber is a collision cell designed to cause fragmentation of ions and to delay extraction. In various embodiments, the fragmentation chamber can also serve as a delayed extraction ion source for the analysis of the fragment ions by time-of-flight mass spectrometry.

In some embodiments, ionization can be used to produce structurally specific fragment ions and Q3 MRM ions. The labeling reagent can be wholly or partly contained in the structurally specific fragment ions. The method can provide both sensitivity and specificity for the Q3 MRM ions. In some embodiments, ionization can be sued to produce a dominant neutral loss fragment ion which can be selected in Q3 and then fragmented to produce structurally specific ions. These fragment ions can then be used for identification and quantification in a procedure referred to as MS3.

Kits

In some embodiments, the present teachings comprise kits for the analysis of ketone or aldehyde analyte compounds. The kit comprises one or more labels, including a set of two or more isotopically enriched standards and one or more reagents, containers, enzymes, buffers and/or instructions for use. Kits of the present teachings comprise one or more sets of supports, each support comprising a different isobaric labeling compound cleavably linked to the support through a cleavable linker. Examples of cleavable linkages comprise, but are not limited to, a chemically or photolytically cleavable linker. The supports can be reacted with different samples thereby labeling the analytes of a sample with the isobaric tag associated with the respective support. Ketone analytes from different samples can be contacted with different supports and thus labeled with different reporter/linker combinations.

According to various embodiments, the kit can comprise a plurality of different aminooxy tagging reagents, for example, a set of labeling reagents as described herein. The kit can be configured to analyze a plurality of different keto or aldehyde analytes, for example, a plurality of different ketosteroids, and the labeling can comprise labeling each with a plurality of different respective labeling reagents, for example, a different reagent for each different type of analyte. The analytes to be analyzed and for which a kit can be configured to detect, can comprise keto or aldehyde compounds, for example, ketosteroids. According to various embodiments of the present teachings, a kit is provided that comprises one or more aminooxy MS tagging reagents for tagging one or more ketone or aldehyde analytes. The aminooxy MS tagging reagent can comprise a compound having one of the structures described herein.

The kit can comprise a standard comprising a known ketone or aldehyde compound, a known steroid, a known ketosteroid, or a combination thereof. The standard can comprise a known concentration of a known compound. In some embodiments, the aminooxy MS tagging reagent comprised in the kit can comprise one or more isobaric tags from a set of isobaric tags. In some embodiments, the kit can comprise a plurality of different isobaric tags from a set of isobaric tags. In some embodiments, the aminooxy MS tagging reagent comprised in the kit can comprise one or more permanently charged aminooxy reagents from a set of permanently charged aminooxy reagents. In some embodiments, the kit can comprise a plurality of different permanently charged aminooxy reagent tags from a set of permanently charged aminooxy reagent tags.

The kit can also comprise instructions for labeling the analyte, for example, paper instructions or instructions formatted in an electronic file, for example, on a compact disk. The instructions can be for carrying out an assay. In some embodiments, the kit can comprise a homogeneous assay in a single container, to which only a sample need be added. Other components of the kit can comprise buffers, other reagents, one or more standards, a mixing container, one or more liquid chromatography columns, and the like.

In some embodiments, a ketosteroid analysis kit is provided that enables highly sensitive quantitation of ketosteroids from complex biological matrices, for example, detection in the range of low pg/mL concentrations.

### DEFINITIONS

The phrases “mass differential labels”, “mass differential tags” and “mass differential labeling reagents” are used interchangeably herein. The phrases “set of mass differential labels”, “set of mass differential tags” are used interchangeably and refer to, for example, a set of reagents or chemical moieties where the members of the set (i.e., an individual “mass differential label” or “mass differential tag”) have substantially similar structural and chemical properties but differ in mass due to differences in heavy isotope enrichment between members of the set. Each member of the set of mass differential tags can produce a different daughter ion signal upon being subjected to ion fragmentation. Ion fragmentation can be, for example, by collisions with an inert gas (e.g., collision induced dissociation (CID), collision a activated dissociation (CAD), etc.), by interaction with photons resulting in dissociation, (e.g., photoinduced dissociation (PID)), by collisions with a surface (e.g., surface induced dissociation (SID)), by interaction with an electron beam resulting in dissociation (e.g., electron induced dissociation (EID), electron capture dissociation (ECD)), thermal/black body infrared radiative dissociation (BIRD), post source decay, or combinations thereof. A daughter ion of a mass differential tag or label that can be used to distinguish between members of the set can be referred to as a reporter ion of the mass differential tag or label.

The phrases “isobaric labels”, “isobaric tags” and “isobaric labeling reagents” are used interchangeably. The phrases “set of isobaric labels”, “set of isobaric tags” and “set of isobaric labeling reagents” are used interchangeably and refer to, for example, a reagents or chemical moieties where the members of the set (an individual “isobaric label,” “isobaric tag,” or “isobaric labeling reagent”) have the identical mass but where each member of the set can produce a different daughter ion signal upon being subjected to ion fragmentation (e.g., by collision induced dissociation (CID), photoinduced dissociation (PID), etc.). A set of isobaric tags comprises compounds of formula (I) or (II), or a salt or a hydrate form thereof. A daughter ion of an isobaric tag that can be used to distinguish between members of the set can be a reporter ion of the isobaric tag or charged analyte. A set of isobaric tags is used to label ketone or aldehyde compounds and produced labeled compounds that are substantially chromatographically indistinguishable, but which produce signature ions following CID. The masses of the individual members of a set of mass labels can be identical or different. Where the individual isotopic substitutions are the same, the masses can be identical. Differences in selecting individual atoms for the heavy or light element incorporated into a specific label of the set can also yield mass differences based on the specific atomic weights of the isotopically enriched substituents.

As used herein, “isotopically enriched” means that a compound (e.g., labeling reagent) has been enriched synthetically with one or more heavy atom isotopes (e.g. stable isotopes including, but not limited to, Deuterium, ¹³C, ¹⁵N, ¹⁸O, ³⁷Cl, or ⁸¹Br). Because isotopic enrichment is not 100% effective, there can be impurities of the compound that are of lesser states of enrichment and these will have a lower mass. Likewise, because of over-enrichment (undesired enrichment) and because of natural isotopic abundance variations, impurities of greater mass can exist.

As used herein, “natural isotopic abundance” refers to the level (or distribution) of one or more isotopes found in a compound based upon the natural terrestrial prevalence of an isotope or isotopes in nature. For example, a natural compound obtained from living plant matter will typically contain about 0.6% ¹³C.

As used herein, the term “salt form” includes a salt of a compound or a mixture of salts of a compound. In addition, zwitterionic forms of a compound are also included in the term “salt form.” Salts of compounds having an amine, or other basic group can be obtained, for example, by reaction with a suitable organic or inorganic acid, such as hydrogen chloride, hydrogen bromide, acetic acid, perchloric acid and the like. Compounds with a quaternary ammonium group may also contain a counteranion such as chloride, bromide, iodide, acetate, perchlorate and the like. Salts of compounds having a carboxylic acid, or other acidic functional group, can be prepared by reacting the compound with a suitable base, for example, a hydroxide base. Accordingly, salts of acidic functional groups may have a countercation, such as sodium, potassium, magnesium, calcium, etc.

As used herein, “hydrate form” refers to any hydration state of a compound or a mixture or more than one hydration state of a compound. For example, a labeling reagent discussed herein can be a hemihydrate, a monohydrate, a dihydrate, etc. Moreover, a sample of a labeling reagent described herein can comprise monohydrate, dihydrate and hemihydrate forms.

As used herein, the term “predominant,” such as “one predominant signature ion fragment” means at least more than 50% of the ions created during the fragmentation process are the signature ions. In some embodiments, at least 60%, 70%, 80%, 90%, of the ions created during the fragmentation process are signature ion. Similarly, the terms predominantly, such as “predominantly neutral loss fragmentation” means at least more than 50% of the ions created during the fragmentation process are neutral loss fragments. In some embodiments, at least 60%, 70%, 80%, 90%, of the ions created during the fragmentation process are neutral loss fragments.

While the above description provides examples and specific details of various embodiments, it will be appreciated that some features and/or functions of the described embodiments admit to modification without departing from the scope of the described embodiments. The above description is intended to be illustrative of the teachings herein, the scope of which is limited only by the language of the claims appended hereto.

### EXAMPLES

Aspects of the applicant's teachings may be further understood in light of the following examples, which should not be construed as limiting the scope of the applicant's teachings in any way.

### Example 1

### Analysis of Biological Samples by LC/ESI/MS/MS

Derivatization Procedure: 200 μL of Te serum was mixed with 200 μL water with 0.1% formic acid and a short pulse vacuum was applied while in a 96=-well plate. After 10 minutes, the sample was eluted twice with 900 μL dichloromethane (DCM) and dried. The analyte was then reacted with the reagent QAO in methanol and acetic acid and incubated for 15 minutes at room temperature. The reaction was 98% complete and had a 93% recovery in solvent and 87% recovery in DCS human serum.

The testosterone was then detected and quantified by internal standard or from a standard curve. A single chromatographic peak at 3.94 was observed. This derivatization is amiable to high throughput automation and takes only 25 minutes. This procedure is also appropriate for other steroids such as progesterone and aldosterone as well as other ketosteroids.

The limit of detection of QAO-Te in DCS human serum was quantified for two different LC protocols: the ‘single peak’ method and the ‘double peak’ method. These two protocols are distinguished by the LC gradient which allows for co-elution (“single peak”) or resolved elution (“double peak”) of the E/Z isomers of QAO Te. The ‘double peak’ method has shallower gradients to assure that the isomers are separated where the single peak method enable co-elution of the isomers of QAO-Te by using a faster gradient.

The ‘double peak’ method used as an example Kinetex (Phenomenex) C18 column (50×2.90, 2.6μ) with a H2O/MeOH/0.1% FA mobile phase. This method provided two resolved peaks where the E and Z isomers eluted at 9.48 and 9.22 minutes. The ‘single peak’ method used a Kromasil C4 column (50×2.0, 3.5μ) with a H2)/acetonitrile/ammonium formate (5 mM)/FA (0.1%) mobile phase. This method provided a single, well-shaped peak eluting at 3.94 minutes.

### Example 2

### Characterization of QAO-Te, AL, Preg, and Prog by FlashQuant™ MALDITrippleQuadropole Mass Spectrometer

The derivatized steroids were diluted in ACN/H₂O before MALDI plate spotting. The steroid sample (S) is mixed with excess matrix (M) and dried on a MALDI plate. The plate is loaded onto the sample stage in the ion source. Laser beam produces matrix neutrals (M), matrix ions (MH)+, (MH)−, and sample neutrals (S). MALDI plate spotting: The steroid sample was mixed with the MALDI matrix α-cyano-4-hydroxycinnamic acid (CHCA) dissolved in ACN/H2O 1/1 V/V+0.1% TFA (10 mg/mL). 0.75 μL spotted on each well and air dried. MALDI Instrument and MRM conditions: Analysis was performed on a 4000 QTRAP® (ABI; Foster City, Calif.) with FlashLaser™ source which is a high repetition laser optimized for the analysis of small molecules. The compound dependent and MRM parameters are described in Table 1. Sample molecules are ionized by proton transfer from matrix ions: MH++S→M+SH+, MH−+S→X+SH−. The FlashLaser™ source, equipped with a high repetition laser, generates ultra fast signal from samples spotted on a target plate.

TABLE I
MALDI-MRM Parameters

| | Laser Power, | MRM | | |
|---|---|---|---|---|
| Steroid | Plate Voltage | Transition | CE (eV) | CXP |
| Testosterone | 15%, 70 V | 289→109 | 35 | 10 |
| (Te) | | | | |
| Derivatized Te | 15%, 70 V | 403→344 | 30 | 9 |
| Aldosterone | 15%, 70 V | 361→325 | 27 | 16 |
| (AL) | | | | |
| Derivatized AL | 15%, 70 V | 475→416 | 35 | 9 |
| Pregnenolone | 15%, 70 V | 317→159 | 47 | 9 |
| (Preg) | | | | |
| Derivatized Preg | 15%, 70 V | 431→372 | 35 | 15 |
| Derivatized Prog | 15%, 70 V | 428→369 | 35 | 10 |
| (As Internal | | | | |
| Standard) | | | | |

### Example 3

### Characterization of QAO-Testosterone in DCS Human Serum ESI/LC/MS/MS API 3200™ LC/MS/MS

A sample of Te in DCS human serum was extracted and derivatized as described above. Chromatographic separation was performed with both the “single peak” and “double peak” methods. The ‘double peak’ method was able to resolve the two E/Z isomers of QAO Te, with elutions at 3.16 and 3.26 minutes; a 10 pg/mL sample provided a S/N ratio of 9 and a LOD of 4 pg/mL. The ‘single peak’ method provided a single, well-shaped peak eluting at 3.92 min; a 10 pg/mL sample provided a S/N ratio of 5.6 and a LOD of 5.5 pg/mL. No detectable peak of QAO Te was found in the blank. For both methods, the reproducibility at 10 pg/mL is less than 10% CV (n=5).

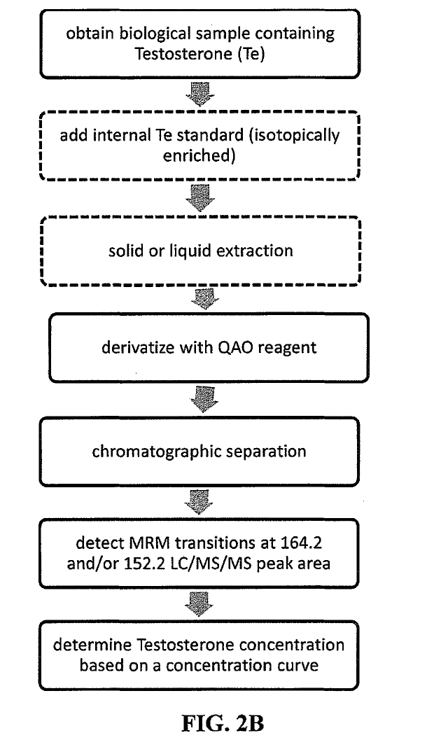

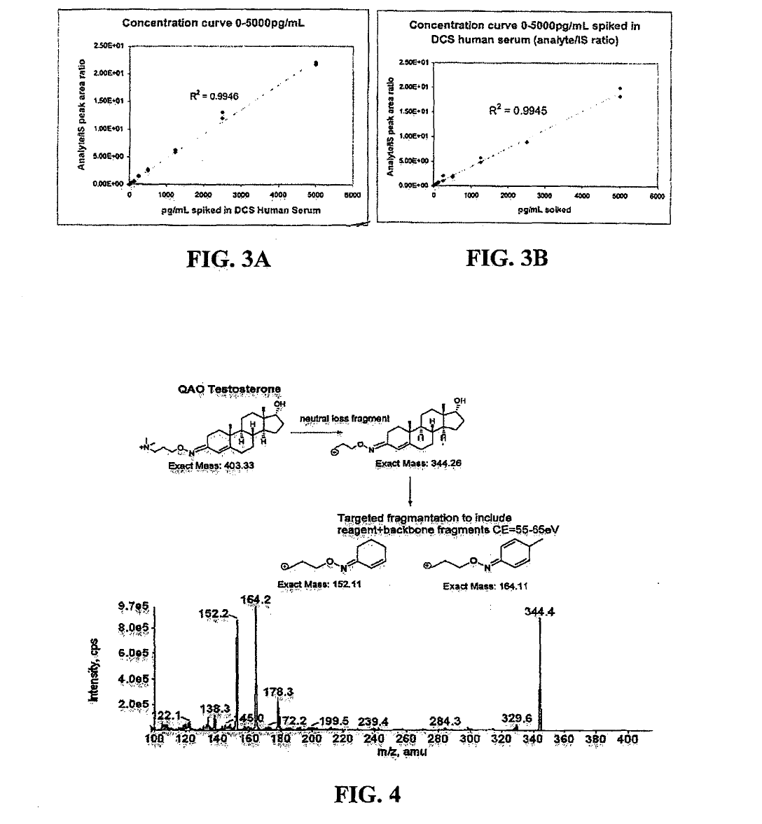

The linearity and dynamic range of QAO-Te in DCS human serum were analyzed. An internal standard of QAO-d₃Te, with an exact mass of 406.4 was used. Both the single peak and double peak methods were analyzed over a range of 0 to 5000 pg/mL spiked in DCS human serum. As shown in FIGS. 3A and 3B, both provided a linear curve.

This range can be broadened by the addition of more reagent.

### Example 4

### Derivatization of Ketosteroids Using Permanently Charged Aminooxy Reagent

Testosterone was derivatized with a QAO reagent according to the methods as described herein and analyzed using MS/MS. FIG. 4 shows the MS/MS fragments and spectrum of QAO Testosterone using CE=62 eV at which the signature ions contain fragments from both testosterone structure and from the derivatizing reagent structure, according to various embodiments of the present teachings.

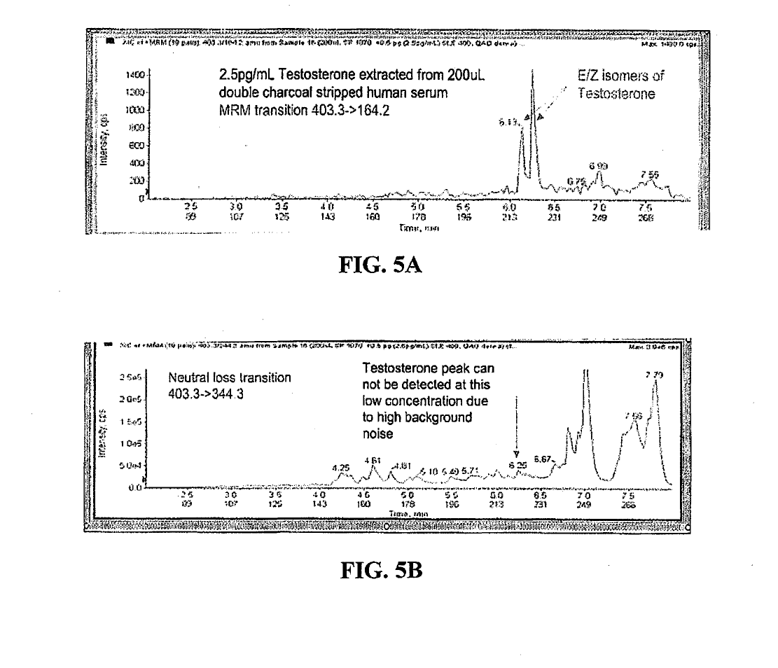

FIGS. 5A and 5B show the chromatograms of QAO derivatized testosterone using an MRM transition of a targeted Q3 fragment as compared to neutral loss Q3 fragment, according to various embodiments of the present teachings. Measurement involved using MRM transitions of neutral loss (403->344, FIG. 5B) vs. the reagent-plus-backbone fragment (304->162, FIG. 5A). As can be seen, lower detection limits are achievable using a Q3 transition that comprises the reagent and the testosterone backbone, due to a significant reduction in background noise.

According to various embodiments of the present teachings, the method is applied to the targeted fragmentation of a ketosteroid. For example, FIG. 6 shows the targeted MS/MS fragmentation and spectrum of QAO Progesterone and the MS/MS spectrum of QAO Testosterone at CE=45 eV. QAO progesterone possesses two keto functionalities and therefore results in bis QAO progesterone.

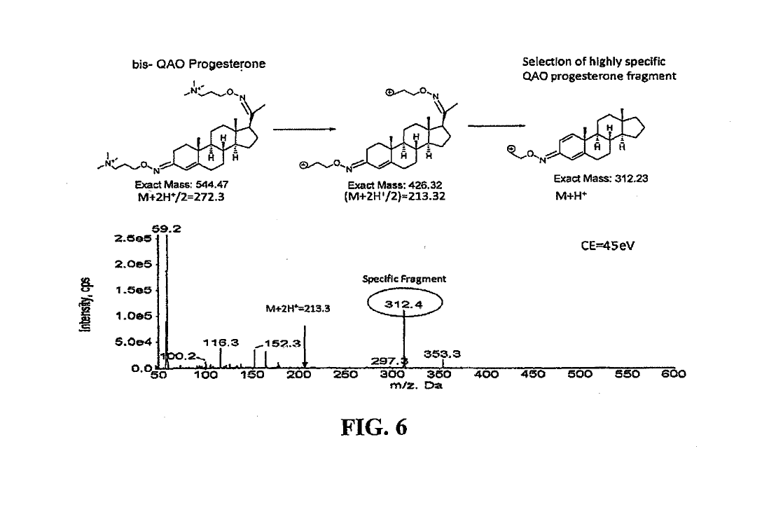

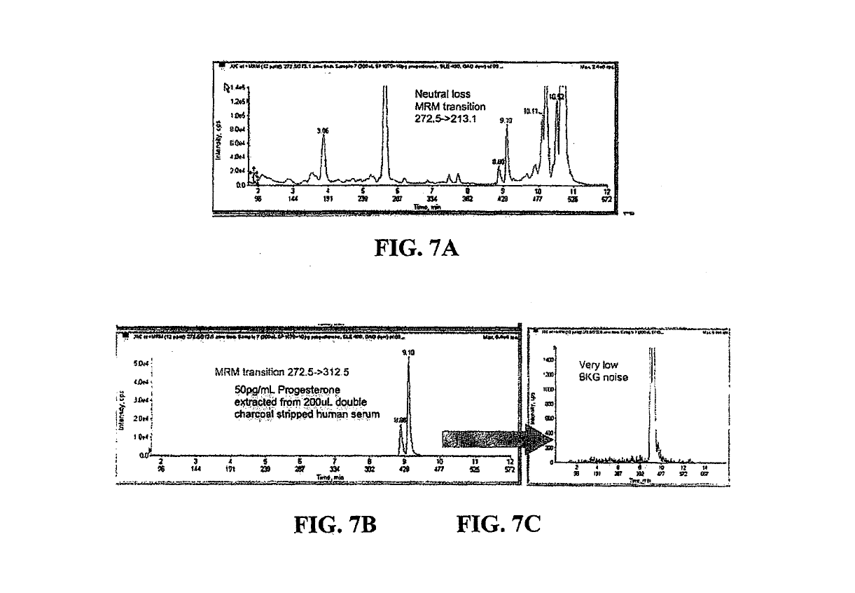

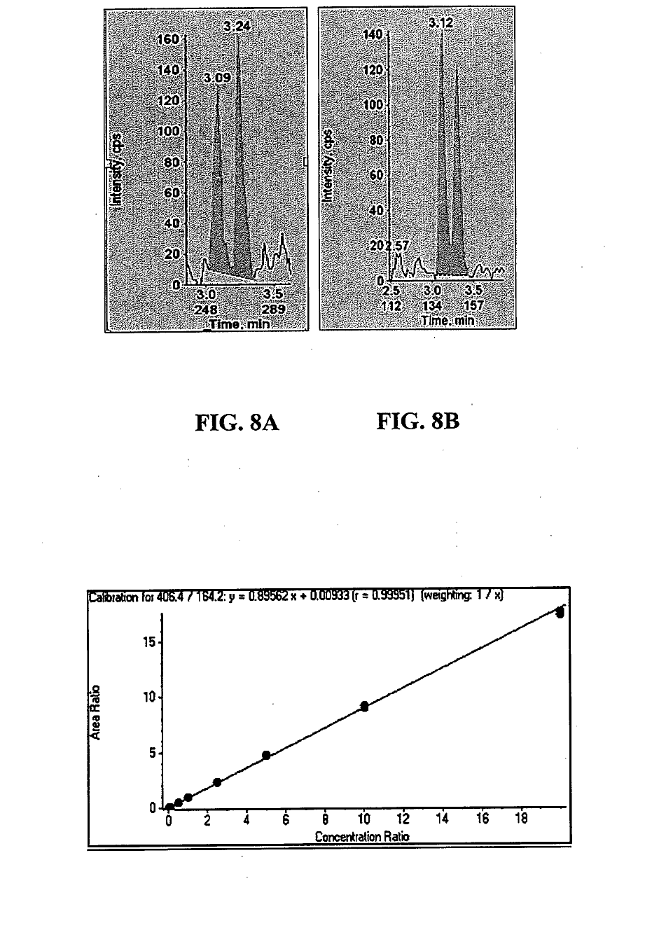

FIGS. 7A-7C show the MS/MS spectrum of progesterone at CE=45 eV. FIG. 7 also illustrates the background noise reduction in an actual LC-MS/MS analysis. The MRM transition 272->213 (FIG. 7B) is the neutral loss from the bis QAO progesterone doubly charged species, and a high background noise is noticeable. The MRM transition of 272->312.5 (FIG. 7A) is the transition from the doubly charged bis QAO to a specific fragment that contains part of the reagent structure and part of the progesterone structure. This MRM transition from a lower Q1 mass to higher Q3 mass is even more specific and further improves specificity and reduces background noise in LC-MRM experiments, showing the very low background noise in the expansion of FIG. 7C.

### Example 5

### Mass Differential and Isobaric Reagents

The following is an exemplary set of quarternary-aminooxy mass differential reagents, according to various embodiments of the present teachings:

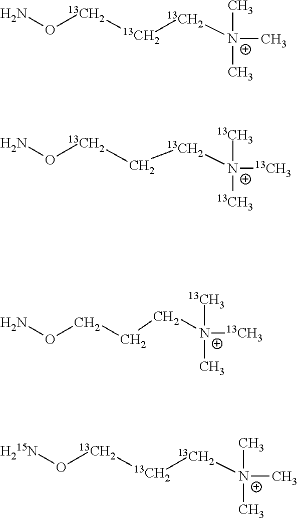

The following is an exemplary set of quarternary-aminooxy isobaric reagents, according to various embodiments of the present teachings:

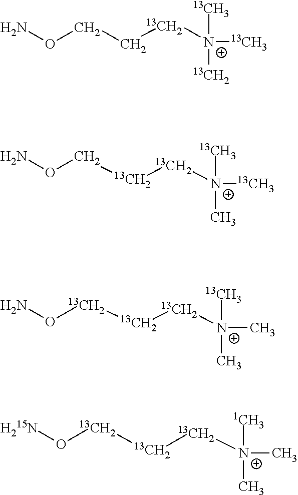

### Example 6

### Testosterone Analysis from Human Serum API 3200™ LC/MS/MS

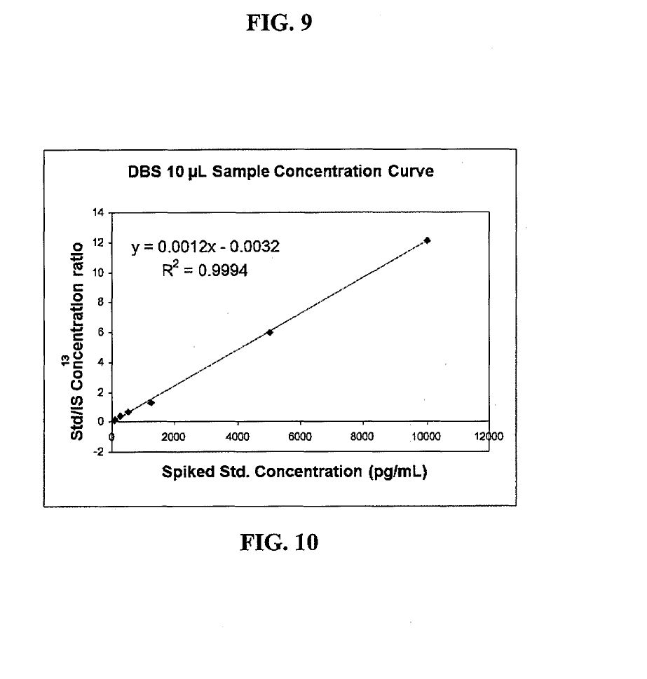

A high sensitivity analysis of testosterone (Te) obtained from human serum samples using QAO derivatization and an API 3200™ LC/MS/MS was performed. ESI/MS/MS sensitivity was found to be enhanced 40 fold upon derivatization. FIG. 9 provides a concentration curve for testosterone between 10 and 10000 pg/mL (200 μL serum, increasing spiked concentrations of d₃Te and 500 pg/mL ¹³C Te IS). The dynamic range covers the reference values of all human samples. Initially 200 μL of human serum/plasma samples were extracted to achieve LLOQ of 10 pg/mL and LOD of 5 pg/mL.

This sample preparation can be performed either by Liquid-Liquid extraction (LLE) or by Solid Liquid Extraction (SLE) with the following workflow:

LLE:
Spike Internal Standard (IS) 100 pg into 200 μL serum/plasma sample
Transfer the 200 μL of serum/plasma sample to a 2 mL Polypropylene vial and spike with isotopically enriched IS (¹³C Te), add 1 mL of extraction solvent (90% Hexane/10% Ethyl acetate).
Vortex mix for 2 min and let stand for 5 min at RT. Centrifuge 5000 rpm for 5 min
Transfer 0.7 mL from the supernatant to a clean 2 mL polypropylene vial
Evaporate the extract to dryness
Reconstitute in 50 μL solution of 10 mg/mL QAO reagent dissolved in MeOH+5% acetic acid.
Vortex mix 45 minutes at RT
Add 20 μL of H₂O (LC/MS grade)
Transfer to a polypropylene HPLC vial for LC/MS/MS analysis.
Inject 154

SLE:
Spike IS (100 pg) into 200 μL serum/plasma sample
Transfer the 200 μL of serum/plasma sample onto a 96 well Solid Phase Extraction plate containing 200 mg Celite in each well.
Wait 5 min and add 1.3 mL Diisopropyl Ether. Let the solvent run through the solid phase for 5 min.
Evaporate the extract to dryness
Reconstitute in 50 μL solution of 10 mg/mL QAO reagent dissolved in MeOH+5% acetic acid.
Vortex mix for 45 minutes at RT,
Add 20 μL H₂O (LC/MS grade)
Transfer the sample to a polypropylene HPLC vial for LC/MS/MS analysis
Inject 15 μL

The LC/MS/MS Conditions are: LC pumps, degasser, autosampler and controller: Agilent 1100 system. Column: Cadenza CL-C18 50×4.6, 3 μm (Imtakt Prod # CL002). Ambient temperature. The mobile phase is:
A=H₂O+0.1% FA+5 mM NH₄COOH
B=Acetonitrile+0.1% FA+5 mM NH₄COOH
Injection volume: 15 μL
Autosampler temperature: Ambient

The gradient is:

| Total Time(min) | Flow Rate(μl/min) | A (%) | B (%) |
|---|---|---|---|
| 0.00 | 800 | 90.0 | 10.0 |
| 0.50 | 800 | 55.0 | 45.0 |
| 1.00 | 800 | 55.0 | 45.0 |
| 3.00 | 800 | 30.0 | 70.0 |
| 3.30 | 800 | 30.0 | 70.0 |
| 4.00 | 800 | 5.0 | 95.0 |
| 4.20 | 2000 | 90.0 | 10.0 |
| 4.80 | 2000 | 90.0 | 10.0 |
| 5.00 | 800 | 90.0 | 10.0 |

The MRM Transitions and MS/MS conditions include a Source Temperature of 600° C. and Ion Spray voltage=4500 V. Additional conditions are shown in Table II.

TABLE II

| Q1 | Q3 | EP | time (msec) | DP | CE | CXP |
|---|---|---|---|---|---|---|
| d₀Te | | | | | | |
| 403.3 | 164.2 | 10 | 200 | 80 | 62 | 10 |
| 403.3 | 152.3 | 10 | 200 | 80 | 60 | 7 |
| d₃Te | | | | | | |
| 406.3 | 164.2 | 10 | 100 | 80 | 62 | 10 |
| 406.3 | 152.3 | 10 | 100 | 80 | 60 | 7 |
| ¹³C Te | | | | | | |
| 406.3 | 167.1 | 10 | 100 | 80 | 62 | 10 |
| 406.3 | 155.1 | 10 | 100 | 80 | 60 | 10 |

### Example 7

### Dried Blood Spots Extraction and Analysis Using QAO Derivatization Using QTRAP® 5500 Applications

The following is a detailed description of Dried Blood Spots (DBS) Extraction and analysis:
Place the dried Blood Spot sample disc containing the whole blood sample (˜8-10 μL) in a 1.5 mL polypropylene vial.
Add 200 μL extraction solvent 90% hexane, 10% Ethyl Acetate and sonicate for 30 min.
Add IS (10 pg/20 μL in MeOH) and evaporate the MeOH extract to dryness. Reconstitute in a 50 μL solution of 10 mg/mL QAO reagent dissolved in MeOH+5% acetic acid.
Vortex mix 45 minutes at RT
Add 20 μL of H₂O (LC/MS grade)
Transfer the sample to a polypropylene HPLC vial for LC/MS/MS analysis
Inject 20 μL

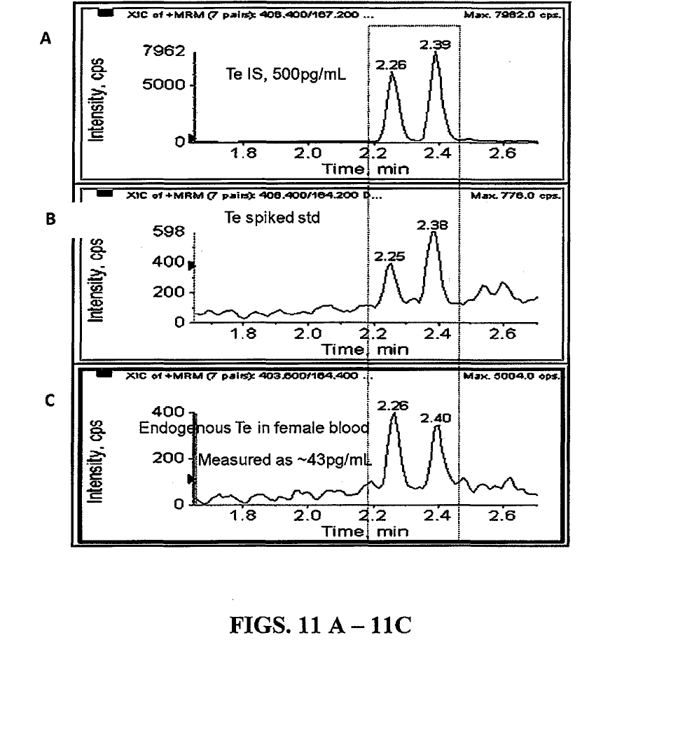

Using the above protocol, a concentration curve for DBS samples is shown in FIG. 10 for a concentration range of 50-1000 pg/mL using d₃Te as calibrant and ¹³C Te as IS. 10 μL of female whole blood was spiked on filter paper disc of ¼″ diameter. FIGS. 11A-11C shows a chromatogram of QAO derivatized female dried blood spot, 10 μL whole blood. (QTRAP® 5500 system). The measurement of its endogenous Te concentration is ˜43 pg/mL. FIG. 11A shows ¹³C Te as internal standard at 500 pg/mL. FIG. 11B shows 50 pg/mL d₃Te spiked. FIG. 11C shows the measured endogenous d₀Te in the DBS sample. The LLOQ using 10 μL whole blood was found to be <50 pg/mL.

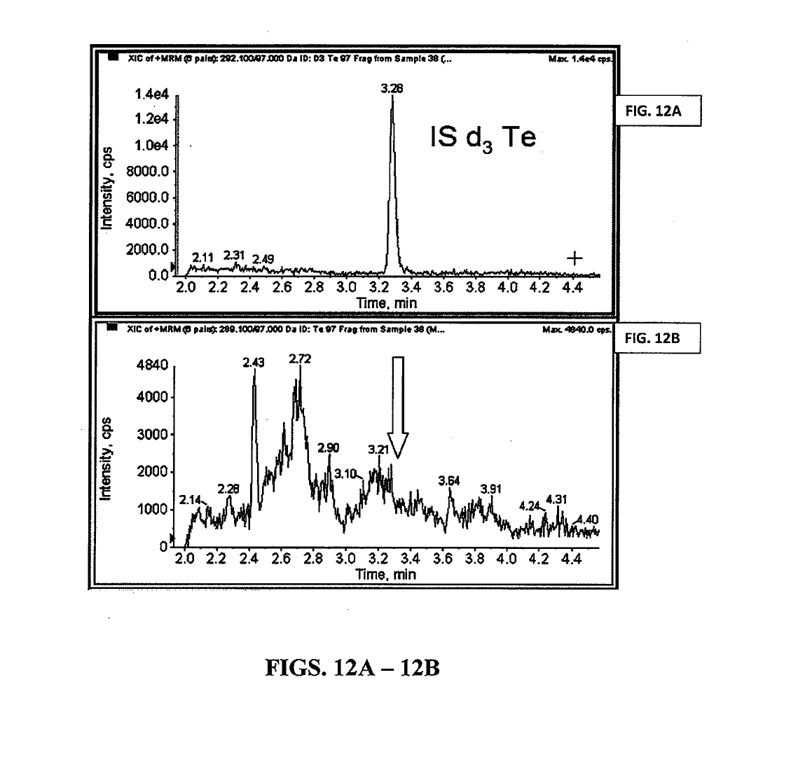

For comparison, FIGS. 12A and 12B show the underivatized DBS from 10 μL female whole blood (same donor presented in FIGS. 11A-11C), using AB SCIEX QTRAP® 5500 System. As shown in FIG. 12B, no signal of underivatized Te could be detected.

### Example 8

### Free Testosterone (FT) Analysis Using QAO Derivatization QTRAP® 5500

The following describes a detailed method of free testosterone (FT) analysis:
Dilute 5004 of Plasma/serum with 500 μL PBS and apply onto a 30 KD MWCO cut off filter (Centrifree YM 30 Ultrafiltration device, Millipore). This membrane retains the protein bound Te (SHBG and Albumin) and allow the FT to pass through the membrane.
Centrifuge the Ultra Filtration device 1-2 h, 2000 g.
Extract 500 μL of the aqueous Ultra Filtrate (UF) by Solid Liquid Extraction (SLE).

The following describes a detailed procedure:
Apply 500 μL of the ultrafiltrate to a 96 well Solid Phase Extraction plate containing 400 mg Celite in each well.
Wait 5 minutes and add 1.5 mL Diisopropyl Ether. Let the solvent run through the solid phase for additional 5 min
Evaporate the extract to dryness
Reconstitute in 50 μL QAO reagent solution 10 mg/mL dissolved in MeOH+5% acetic acid.
Vortex mix 45 minutes at RT,
Add 20 μL of H₂O (LC/MS grade)
Transfer the sample to a polypropylene HPLC vial for LC/MS/MS analysis.

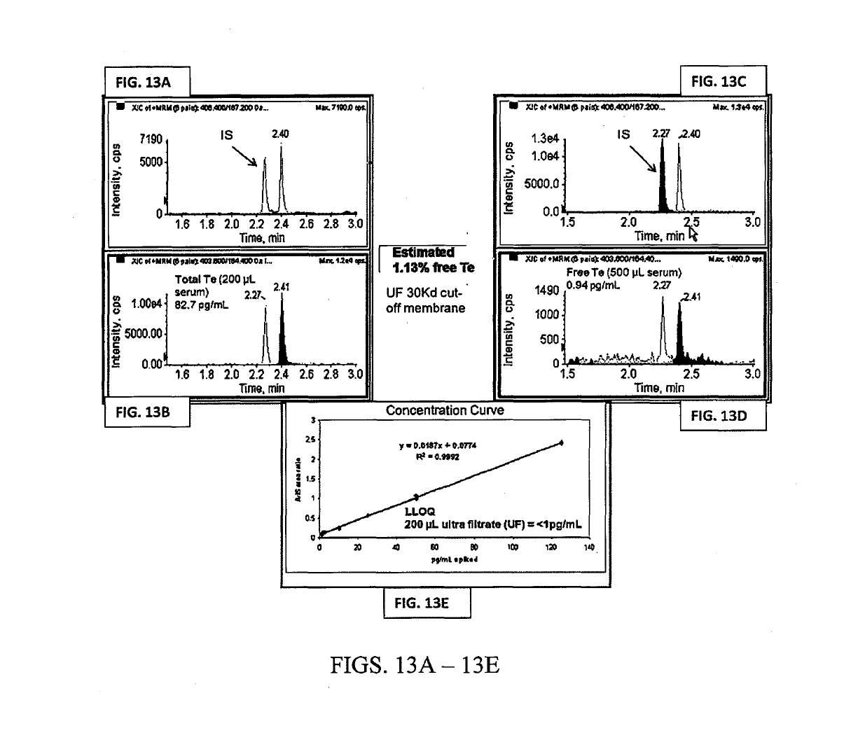

FIG. 13 provides an analysis of a sample containing free testosterone from female serum (pool) using the above procedure with QAO derivatization and QTRAP® 5500 Instrument. ¹³C Te was used as IS and d₃Te spiked was used as a calibrant in the concentration curve.

The LLOQ using 500 μL serum was found to be ˜0.5 pg/mL.

### Example 9

### Extraction of Free Testosterone from Saliva Samples Using QAO Derivatization QTRAP® 5500

Human saliva contains only free Te. This is an easy sample to obtain and the extraction procedure is simple. The following described a detailed procedure:
Pipette 1 mL saliva into microcentrifuge polypropylene vial
Add 1 mL extraction solvent 90% Hexane/10% ethyl acetate and 20 pg isotopically enriched IS (¹³C or d₃Te)
Vortex mix for 5 min
Centrifuge 5 min, 14000 rpm
Remove 500 μL from the supernatant
Dry and reconstitute in 50 μL solution of 10 mg/mL QAO reagent dissolved in MeOH+5% acetic acid.
Vortex mix 45 minutes at RT,
Add 20 μL of H₂O (LC/MS grade)
Transfer the sample to a polypropylene HPLC vial for LC/MS/MS analysis.

FIG. 13 shows an estimate of free testosterone concentration in female saliva (1 mL) using the above procedure with QAO derivatization and QTRAP® 5500 Instrument. One point calibration, 20 pg/mL d₃Te was used as IS.

The LC/MS/MS Conditions: QTRAP® 5500 are as follows: LC pumps, degasser, autosampler and controller: Shimadzu Nexera 30A system. Column: Cadenza CL-C18 50×4.6, 3 μm (Imtakt Prod # CL002). Ambient temperature. The mobile phase contained:
A=H₂O+0.1% FA B=Acetonitrile+0.1% FA Injection volume: 20 μL
Autosamplertemperature: Ambient

The gradient was according to the following table

TABLE III

| Time | % A | % B | Flow (mL/min) |
|---|---|---|---|
| 0 | 90 | 10 | 0.8 |
| 0.5 | 70 | 30 | 0.8 |
| 1 | 70 | 30 | 0.8 |
| 4 | 35 | 65 | 0.8 |
| 4.3 | 35 | 65 | 0.8 |
| 5 | 5 | 95 | 0.8 |
| 5.2 | 90 | 10 | 2 |
| 5.8 | 90 | 10 | 2 |
| 6 | 90 | 10 | 0.8 |

A Valco valve diverted the first 1.5 minutes to waste.

The ESI/MS/MS Conditions were as follows: Source temperature=650° C. and Ion spray voltage=3500 V. Additional parameters are provided in Table IV.

TABLE IV

| Analyte/IS | Q1 | Q3 | EP | time (msec) | DP | CE | CXP |
|---|---|---|---|---|---|---|---|
| d0 Te_1 | 403.6 | 164.4 | 10 | 100 | 50 | 62 | 9 |
| d0 Te_2 | 403.6 | 152.2 | 10 | 100 | 50 | 60 | 9 |
| d3 Te_1 | 406.4 | 164.2 | 10 | 100 | 50 | 62 | 9 |
| d3 Te_2 | 406.4 | 152.4 | 10 | 100 | 50 | 60 | 9 |
| 13C Te_1 | 406.4 | 167.2 | 10 | 100 | 50 | 62 | 9 |
| 13C Te_2 | 406.4 | 155.2 | 10 | 100 | 50 | 60 | 9 |

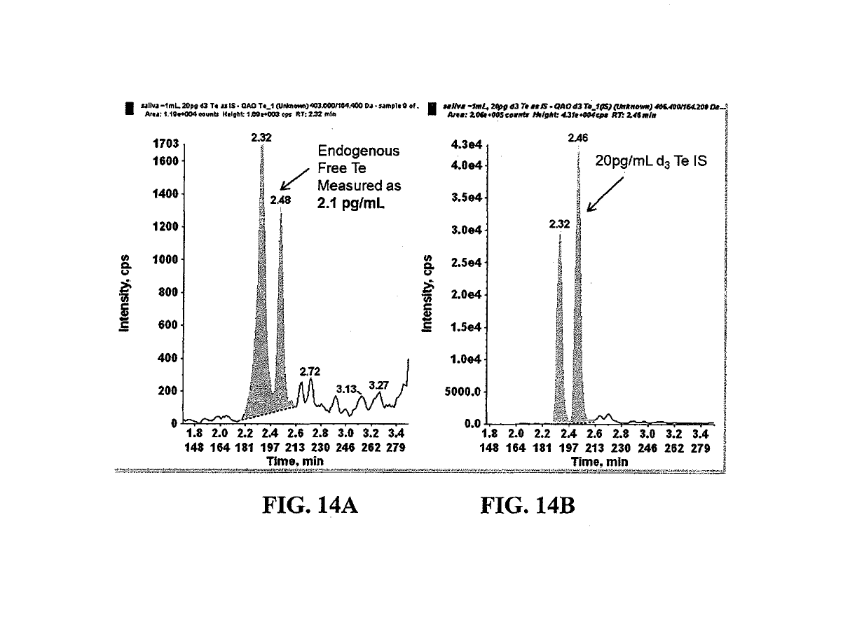

FIGS. 14A and 14B show the endogenous free testosterone. The sample provided a concentration of 2.1 pg/mL (FIG. 14A). FIG. 14B provides a 20 pg/mL of d₃ testosterone internal standard.

Other examples of suitable MRM transitions for various types of ketosteroids containing one, two and three keto groups are listed in Tables V, VI, VII below, respectively. In these tables, NL=Neutral loss, −59 of Trimethylamine which can also lose −118 if the bis-derivative is formed. For bis derivatives a multiply charged fragment is detectable in Q1 (MRM 1) and a singly or doubly charged Fragment in Q2 (MRM 2). In the latter scenario, the Q3 mass is higher as an absolute number (e.g. Progesterone: MRM transition 272.5->312.5). These MRM transitions of higher “mass” in Q3 are more specific in nature. Another common type of bis ketosteroids fragment is the loss of only one Trimethylamine. If the MRM transition reflects a doubly charged parent ion converting to another doubly charged product ion, which is a loss of one trimethylamine, the absolute loss is −30 (e.g. 11 deoxy cortisol MRM transition 288.5->258.9)

TABLE V

| | Mass | Mass | MRM Transitions |
|---|---|---|---|
| Ketosteroid | Underivatized | Derivatized | Mono derivatized |
| DHEA | 288.21 | 403.33 | 403.3->91.1 |
| (Dehydroepiandrosterone) | | | 403.3->344.3 (NL) |
| | | | 403.3->156.9 |
| | | | 403.3->141.2 |
| | | | 403.3->328.2 |
| | | | 403.3->128.2 |
| | | | 403.3->253.2 |
| DHEAS | 368.17 | 483.29 | 483.29->326.3 |
| (Dehydroepiandrosterone- | | | 483.29->424.4 (NL) |
| sulfate) | | | 483.29->344.4 |
| | | | 483.29->142.1 |
| | | | 483.29->128.2 |
| Pregnenolone | 316.24 | 431.36 | 431.4->372.3 |
| | | | 431.4->126.3 |
| 17 Hydroxy pregnenolone | 332.47 | 447.36 | 447.7->388.4 (NL) |
| | | | 447.7->370.4 (NL-18) |
| | | | 447.7->144.3 |
| | | | 447.7->352.1 |
| Allopregnanolone | 318.26 | 433.38 | 433.5->374.3 (NL) |
| | | | 433.5->126.1 |
| | | | 433.5->100.1 |
| Testosterone | 288.21 | 403.30 | 403.3->344.3 (NL) |
| | | | 403.3->164.2 |
| | | | 403.3->152.2 |
| Epi-testosterone | 288.21 | 403.30 | 403.3->344.3 (NL) |
| | | | 403.3->164.2 |
| | | | 403.3->152.2 |
| Dihydrotestosterone (DHT) | 290.22 | 405.35 | 405.35->346.3 (NL) |
| Estrone | 270.16 | 385.28 | 385.3->326.2 (NL) |
| | | | 385.3->253.3 |
| | | | 385.3->157.1 |

TABLE VI

| | Mass | Mass | MRM Transitions | MRM Transitions |
|---|---|---|---|---|
| Ketosteroid | Underivatized | Derivatized | Mono derivatized | Bis derivatized |
| 17 Hydroxyprogesterone | 330.2 | Mono = 445.34 | 445.6->386.4 (NL) | 280.5->328.3 |
| (17 OHP) | | Bis = 560.47 | 445.6->164 | 280.5->250.7 |
| | | (doubly | 445.6->152.2 | |
| | | charged = 280.24) | | |
| 11-deoxycorticosterone | 330.2 | Mono = 445.6 | 445.6->386.3 (NL) | 280.3->353.4 |
| (11-DOC) | | Bis = 560.47 | 445.6->152.1 | 280.3->250.3 |
| | | doubly charged = | 445.6->164.1 | |
| | | 280.23 | 445.6->178.3 | |
| Progesterone | 314.2 | Mono = 429.5 | 429.5->164.2 | 272.5->312.5 |
| | | Bis = 544.47 | 429.5->152.3 | 272.5->353.2 |
| | | doubly charged = | | 272.5->164.1 |
| | | 272.5 | | 272.5->152 |
| Cortisol (F) | 362.2 | Mono = 477.33 | 477.5->418.3 (NL) | 296.5->266.8 |
| | | Bis = 592.46 | 477.5->388.1 | 296.5->385.3 |
| | | doubly charged = | 477.5->358.3 | 296.5->344.2 |
| | | 296.5 | | 296.5->237.2 |
| 11 deoxycortisol | 346.21 | Mono = 461.3 | 461.4->402.2 (NL) | 288.5->258.9 |
| (Substance S) | | Bis = 576.46 | 461.4->372.3 | 288.5->369.2 |
| | | doubly charged = = | 461.4->342.3 | |
| | | 288.23 | 461.4->164.1 | |
| | | | 461.4->152.1 | |
| Corticosterone | 346.21 | Mono = 461.3 | 461.4->178.3 | 288.5->369.1 |
| (17-deoxycortisol ) | | Bis = 576.46 | 461.4->203.1 | 288.5->328.2 |
| | | doubly charged = | 461.4->164 | |
| | | 288.23 | 461.4->152 | |
| | | | 461.4->328.6 | |
| 21 Deoxycortisol (21-F) | 346.21 | Mono = 461.3 | 461.4->402.2 (NL) | 288.5->258.7 |
| | | Bis = 576.46 | 461.4->152.1 | 288.5->344.2 |
| | | doubly charged = = | 461.4->178.2 | 288.5->178.2 |
| | | 288.23 | | |
| Androstenedione | 286.19 | Mono = 401.32 | 401.32->164.2 | 258.2->199.2 |
| | | Bis = 516.44 | 401.32->152.1 | 258.2->228.6 |
| | | doubly | | 258.2->286.3 |
| | | charged = 258.22 | | 258.2->340.1 |
| | | | | 258.2->232 |

TABLE VII

| | | | MRM |
|---|---|---|---|
| | | | Transitions |
| | Mass | | Mono |
| Ketosteroid | Underivatized | Mass Derivatized | derivatized |
| Aldosterone | 360.19 | Mono: 475.32 | 475.6->416.5 |
| Only one keto group is | | | (NL) |
| derivatized | | | 475.6->203.3 |
| | | | 475.6->178.3 |
| | | | 475.6->152.1 |
| | | | 475.6->164.1 |

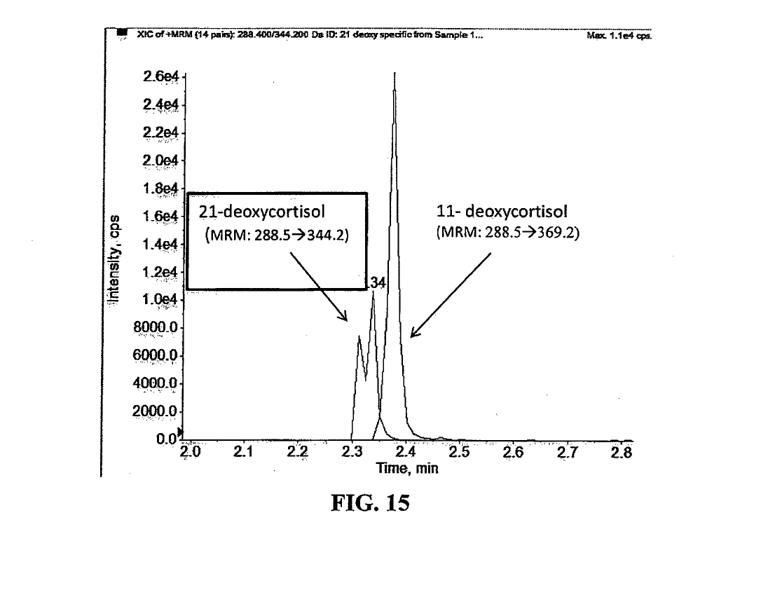

FIG. 15 depicts LC/MS/MS Chromatograms of isobaric ketosteroids using structural specific fragments of the MRM transitions for 21-deoxycortisol and 11-deoxycortisol. Other chromatograms for other ketosteroids can be visualized using data from the tables above.

All literature and similar material cited in this application, including, but not limited to, patents, patent applications, articles, books, treatises, and web pages, regardless of the format of such literature and similar materials, are expressly incorporated by reference in their entireties for all purposes. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

The teachings should not be read as limited to the described order or elements unless stated to that effect. It should be understood that various changes in form and detail may be made without departing from the scope of the present teachings. By way of example, any of the disclosed method steps can be combined with any of the other disclosed steps to provide a method of analyzing ring-containing compounds in accordance with various embodiments of the present teachings. Therefore, all embodiments that come within the scope and spirit of the present teachings and equivalents thereto are claimed.

1. A method for mass analysis of an analyte from a biological matrix comprising:
derivatizing an analyte comprising an aldehyde or ketone functional group, with a labeling reagent of formula (I):
Y—(CH₂)ₙ—O—NH₂ (I)
where n is 2, 3, 4, 5, or 6 and Y is: 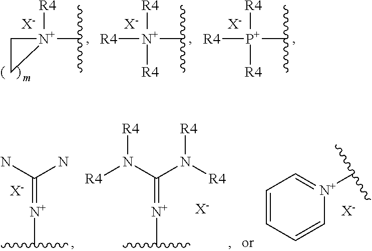
each R₄ is independently H or a C₁-C₁₈ alkyl which is branched or straight chain,
m is an integer between 1 and 20, and
X is an anion,
or a salt or hydrate thereof, to form a labeled analyte;
ionizing the labeled analyte at a low collision energy so as to produce one predominant signature ion fragment; and
detecting the signature ion fragment by mass analysis.

2. The method of claim 1, wherein said signature ion fragment is a neutral loss fragment comprising a structural fragment of the analyte and the labeling reagent or a part thereof.

3. The method of claim 1, wherein said method further comprise the step of extracting said analyte using either liquid-liquid extraction solid-liquid-extraction or protein precipitation prior to said derivatizing step.

4. The method of claim 1, wherein said method further comprises the step of subjecting said analyte to chromatographic separation prior to said derivatizing step.

5. The method of claim 1, wherein the analyte comprises an aldehyde or ketone functional group in a steroid.

6. The method of claim 1, further comprising the step of derivatizing a standard compound with a labeling reagent of formula (I) to form a labeled standard, wherein the labeled standard is isotopically enriched, and ionizing both the labeled analyte and the labeled standard.

7. The method of claim 6, wherein the isotopically enriched labeled standard comprises at least two heavy atoms.

8. The method of claim 6, further comprising measuring the relative concentration of the labeled analyte relative to that of the labeled standard compound.

9. The method of claim 1, further comprising determining said analyte concentration based on a concentration curve.

10. The method of claim 1, wherein the collision energy is in the range of about 30 to about 130 ev.

11. The method of claim 1, wherein the labeling reagent has the structure: 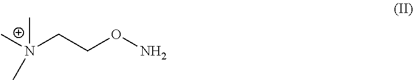
or a salt or hydrate thereof.

12. The method of claim 10, wherein the signature ion fragment comprises: 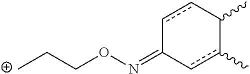
wherein each [symbol] is either a single or double bond and each [symbol] is either absent or indicates one or more bonds.

13. The method of claim 12, wherein the analyte is a testosterone or testosterone derivative and a signature ion fragment has a mass of 152.11 and a second signature ion fragment has a mass of 164.11 or the analyte is a progesterone or progesterone derivative and the signature ion fragment has a mass of 312.23.

14. The method of claim 1, wherein at least two different analytes are derivatized, ionized, and detected.

15. A kit for analysis of ketosteroids comprising a set of mass labels comprising two or more compounds of formula (I):
Y—(CH₂)ₙ—O—NH₂ (I)
where n is 2, 3, 4, 5, or 6 and Y is: 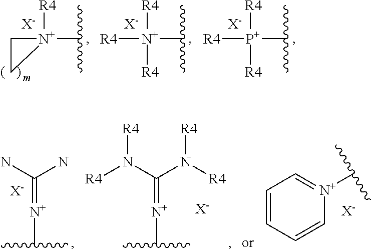
each R₄ is independently H or a C₁-C₁₈ alkyl which is branched or straight chain,
m is an integer between 1 and 20, and
X is an anion,
or a salt or hydrate thereof,
and one or more or a buffer, a reagent, a separation column, and instructions for carrying out an assay.
